(19) United States
(12) Patent Application Publication
(10) Pub. No.: US 2013/0058988 A1
(43) Pub. Date: Mar. 7, 2013

(54) Metal Cations and Metal Effect Pigments Comprising Anions Containing Phosphorus and/or Sulphur, Method for Producing Said Metal Effect Pigments and Use Thereof

(71) Applicants: Pär Winkelmann, Ruckersdorf (DE); Phu Qui Nguyen, Monchengladbach (DE)

(72) Inventors: Pär Winkelmann, Ruckersdorf (DE); Phu Qui Nguyen, Monchengladbach (DE)

(73) Assignee: ECKART GMBH, Hartenstein (DE)

(21) Appl. No.: 13/697,539

(22) PCT Filed: May 10, 2011
(86) PCT No.: PCT/EP11/02318

§ 371 Date: Nov. 13, 2012

(30) Foreign Application Priority Data
May 14, 2010 (DE) 10 2010 020 507.9

(51) Int. Cl. C09C 1/64; C09D 1/00; A61K 33/34; A61K 8/26; A61K 33/06; C09C 1/66; A61K 8/25

(52) U.S. Cl. 424/400; 106/404; 106/403; 106/286.5; 106/286.7; 424/682; 424/630

(57) ABSTRACT

The invention relates to a metallic effect pigment selected from platelet-shaped aluminum, platelet-shaped metallic pigments having a copper fraction of 60% to 100% by weight, and mixtures thereof, having a coating of silicon oxide SiOx, where x is a number from 1 to 2, where the coated metallic effect pigment comprises metal cations and phosphorus- and/or sulfur-containing anions present independently of one another on the metallic effect pigment surface and/or in the silicon oxide layer SiOx, and the element ratio in atomic fractions of metal cation MC and phosphorus P and/or sulfur S to silicon Si is defined by formulae (I) and (II):

100%×(MC+P)/Si (I)

and/or

100%×(MC+S)/Si (II)
and being in total in a range from 0.5% to 35%. The invention further relates to the coated metallic effect pigments producible via the method of the invention, and to the use thereof.

The present invention relates to metallic effect pigments coated by sol-gel processes with silicon oxide, with metal cations and phosphorus- and/or sulfur-containing anions being present on the metallic effect pigment surface and/or in the silicon oxide layer. The invention further relates to a method for producing these metallic effect pigments, and also to the use thereof.

The meteoric rise in the use of eco-friendly waterborne paints in the automobile segment and in industrial coatings has necessitated the development of corrosion-protected metallic effect pigments. In the waterborne paints, which are given a basic formulation, the widespread aluminum effect pigments, in particular, exhibit a propensity toward oxidation, which produces hydrogen and leads to oxidation of the aluminum effect pigment. This oxidization results in a loss of specular gloss, such loss also being referred to as graying. The hydrogen formed constitutes a considerable hazard potential, particularly since a possible oxyhydrogen gas explosion may also lead subsequently to combustion of the finely divided metallic pigments.

Very effectively corrosion-protected aluminum effect pigments are produced by the chromating process (EP 0 259 592) and are available from Eckart under the trade name Hydrolux®. These corrosion-protected aluminum effect pigments are notable for excellent gassing stability and outstanding opacity. Opacity, also called hiding power, is understood as being the surface area hidden per unit weight of pigment. The opacity of the chromated aluminum effect pigments is more particularly comparable with the opacity of the aluminum effect pigments prior to chromating.

A disadvantage is that the chromated aluminum effect pigments contain chromium compounds. Although chromated aluminum effect pigments do not contain any detectable amounts of the toxic Cr(IV), they are nevertheless not advantageous environmentally in view of the heavy metal content.

For this reason, SiO₂-coated aluminum or gold bronze effect pigments were developed. Coating with SiO₂ is accomplished preferably using the sol-gel process, in which, first of all, the aluminum or gold bronze effect pigments undergo sol-gel encapsulation, and subse-quently a silicon dioxide coating is formed. The SiO₂-coated aluminum or gold bronze effect pigments have a high corrosion resistance, since the barrier effect of the silicon dioxide coating prevents the migration of water or other corrosive substances at the pigment surface.

SiO₂ coating takes place via a gentle, eco-friendly sol-gel process which is catalyzed by bases (A. Kiehl, K. Greiwe Progr. in org. Coatings 37 (1999), 179-183). Commercially available metallic effect pigments SiO₂-coated using sol-gel processes are Hydrolan® (aluminum effect pigments) and Dorolan® (gold bronze effect pigments) from Eckart. Other commercially available SiO₂-coated aluminum effect pigments are, for example, Emeral® from Toyo, Japan, Aquamet® from Schlenk, Germany, and Silbercote® from Silberline, USA.

The gassing stability possessed by SiO₂-coated aluminum effect pigments is generally sufficient. By sufficient gassing stability is meant that under the influence of water there is generally no substantial evolution of hydrogen, since the aluminum is protected relatively effectively against attack by water. The gassing stability, however, is also dependent on the ambient conditions to which the aluminum effect pigment is exposed.

Occasionally, in the case of metallic effect pigments having a very fine particle-size band and a corres-pondingly large specific surface area, i.e., surface area per unit weight of metallic effect pigment, unwanted fluctuations may occur in the gassing stability. The opacity of the SiO₂-coated aluminum effect pigments known from the prior art, moreover, decreases markedly, deleteriously, as compared with the starting material and in comparison to chromated pigments.

For the coating of substrates with silicon dioxide there are two methods described in the prior art as being essential. The first method is the utilization of alkali metal silicates, which are converted into silanols by catalyzed hydrolysis before subsequently coalescing to form an inorganic network (R. K. Iler et al U.S. Pat. No. 2,885,366, 1959; R. K. Iler “The Chemistry of Silica”, 1979).

The second method is the utilization of the sol-gel process, starting from alkoxysilanes, which are reacted under catalysis with water to form silanol and alcohol. In the conventional sol-gel coating of aluminum effect pigments using alkoxysilanes, the starting pigment in powder form is dispersed in an alcoholic phase and then the alkoxysilanes, water, and at least one basic or acidic catalyst are added with accompanying supply of heat.

The hydrolysis of tetraethoxysilane compounds is accompanied by formation of silanol structures of the composition Si(OH)_{4-y}(OCH₂CH₃)_{y} (y=0-3), which are able to enter into polycondensation reactions. In the course of the reaction, a compact network of silicon dioxide develops on the surface of the pigment and completely encapsulates the pigment particles. Furthermore, the silicon dioxide coating freshly precipitated onto the pigment surface can be specifically subjected to further surface modifications. For example, silanes having at least one nonhydrolyzable substituent, examples being alkylsilanes, can be added after the application of the SiO₂ coating and can be hydrolyzed in situ, with the silanes having at least one non-hydrolyzable substituent being firmly anchored, via further condensation reactions, to and on the silicon dioxide layer on the pigment surface. The filtercake which is obtained after cooling and suction removal of the solution can be dried under reduced pressure and supplied for the use as intended.

U.S. Pat. No. 2,885,366 A discloses a base-catalyzed method for producing a product surface-stabilized by with metal oxides, it also being possible for this product to consist of SiO₂-coated metallic effect pigments.

Production of effect pigments coated with reactive orientation assistants—using a basic catalyst—is described in DE 198 20 112 A1.

Waterborne basecoat materials comprising SiO₂-coated aluminum effect pigments are disclosed in EP 1 332 714 A1.

WO 2004/026268 A2 as well discloses a method for producing a corrosion-stable metallic effect pigment for a cosmetic product, which involves providing the aluminum core with an SiO₂ coating by means of a sol-gel process using suitable catalysts.

EP 1 756 234 B1 relates to a method for producing an aqueous coating composition which comprises at least one water-compatible, film-forming agent and aluminum effect pigments provided with at least one inorganic corrosion protection layer. These aluminum effect pigments have at least one SiO₂ layer produced by a sol-gel process. No details relating to the stabilization with respect to corrosion are evident from that patent.

WO 03/014228 A1 discloses platelet-shaped aluminum effect pigments which are pretreated with free phosphoric acid and/or boric acid and are then provided with an SiO₂ layer by means of a sol-gel process in organic solvent.

WO 2006/066825 A2 describes a pigment with a similar construction, further comprising an outer layer of tin oxide.

DE 198 36 810 A1 describes multilayer effect pigments based on metal substrate platelets. Coating operations take place in aqueous medium, and for this reason the pigments must be passivated beforehand.

DE 44 37 753 A1 as well discloses multilayer effect pigments based on metal substrate platelets. In this case an SiO₂ layer may be applied, inter alia by means of sol-gel processes. Beforehand the metal platelets may be passivated.

EP 1 619 222 A1 relates to a process for producing an aluminum effect pigment provided with a molybdenum coating and/or SiO₂ coating and intended for water-based inks, using organic bases, such as ethanolamine, for example, or organic or inorganic acids, such as sulfuric acid or oxalic acid, for example, as catalysts.

WO 03/095564 A1 relates to a process for producing goniochromatic luster pigments having a coating which displays interference colors, with a polar organic solvent being incorporated into the coating. To produce these goniochromatic luster pigments, the pigment particles, such as, for example, corrosion-stabilized aluminum effect pigments, are first coated with a dielectric layer of low refractive index, such as silicon dioxide, for example, and are subsequently provided with a reflective coating.

DE 100 01 437 A1 discloses a semifinished product where a prestabilized metallic effect pigment is dispersed with anticorrosion pigments in water. The gassing stability of these products, however, is not very high, and the addition of the anticorrosion pigments may result in incompatibilities with the paint system to be used.

U.S. Pat. No. 5,348,579 and U.S. Pat. No. 5,36,469 as well disclose metallic effect pigments which are treated with anticorrosion pigments. The pigments, however, do not have an SiO₂ coating, and their gassing stability is inadequate.

WO 2004/092284 A1 discloses effect pigments having calcined metal oxide layers, which may be present in a mixture with phosphates. If metallic effect pigments are used as substrate, the resultant coated metallic effect pigment cannot be calcined, for safety reasons. At the customary calcining temperatures, the melting points of the metals, which in the case of aluminum, for example, is 630° C., may be exceeded. Furthermore, with aluminum in particular, there is a great risk, at high temperatures, that the known, strongly exothermic, aluminothermic reaction with the applied metal oxide layers may take place, a reaction which occurs readily particularly with the high specific surface areas and the intimate contact between the layers.

U.S. Pat. No. 6,379,804 B1 discloses coatings comprising metallic effect pigments clad with pure SiO₂ and selected from a group consisting of nickel, nickel alloy, iron, iron alloy, gold, gold alloy, silver, silver alloy, and platinum and platinum alloy. Provided beneath the pure SiO₂ layer there may be a separate thin phosphate layer, applied directly to the metal pigment surface using triethyl phosphate.

The known processes for the SiO₂ coating of metallic effect pigments have the disadvantage, however, that they do not always—especially in the case of very fine metallic effect pigments—ensure sufficient corrosion stability, more particularly gassing stability, of the metallic effect pigments obtainable therewith. There is an ongoing need to increase further the gassing stability of the metallic effect pigments. Moreover, there is usually a distinct loss of opacity as a result of the SiO₂ coating.

Metallic effect pigments whose metals are selected from the group consisting of aluminum, copper, and alloys thereof are very sensitive toward corrosion. Accordingly, aluminum, copper, and also alloys thereof, such as brass or bronze, for example, are easily oxidized by water and/or harsh ambient conditions. Extremely undesirably, oxidation of these metals impairs the optical properties of these metallic effect pigments.

It is an object of the invention to provide metallic effect pigments whose metals are selected from the group consisting of aluminum, copper, and alloys thereof and which are stabilized with respect to corrosion and exhibit improved performance properties. The intention more particularly is for the metallic effect pigments to be provided to exhibit enhanced gassing stability and/or opacity. At the same time, the optical properties of the metallic effect pigments, such as luster and light-dark flop, ought not to be largely impaired.

The object on which the invention is based is achieved by providing a metallic effect pigment selected from the group consisting of platelet-shaped aluminum, platelet-shaped metallic pigments having a copper fraction of 60% to 100% by weight, and mixtures thereof, having a coating of silicon oxide SiOₓ, where x is a number from 1 to 2, where the coated metallic effect pigment comprises firstly metal cations and secondly phosphorus- and/or sulfur-containing anions, the metal cations and phosphorus- and/or sulfur-containing anions being present in each case independently of one another on the metallic effect pigment surface and/or in the silicon oxide layer SiOₓ, and the element ratio in atomic fractions of metal cation MC and phosphorus P and/or sulfur S to silicon Si being defined in each case in accordance with the formulae (I) and (II)

100%×(MC+P)/Si (I)

and/or

100%×(MC+S)/Si

and being situated in total in a range from 0.5% 35%.

Preferred developments are specified in dependent claims 2 to 17.

It is a further object of the invention to provide a method for producing metallic effect pigments whose metals are selected from the group consisting of aluminum, copper, and alloys thereof, featuring enhanced opacity and gassing stability.

The object on which the invention is based is further achieved by providing a method for producing silicon oxide-coated metallic effect pigments, where the method comprises the following steps:
(a) applying silicon oxide to the metallic effect pigments, with reaction of alkoxysilane(s) and/or silicon halide(s) in organic solvent with water in the presence of an acid and/or base as catalyst,
(b) adding phosphorus- and/or sulfur-containing anions and metal cations to the metallic effect pigments before or during step (a), with phosphorus- and/or sulfur-containing anions and metal cations being taken up onto the metallic effect pigment surface and/or into the silicon oxide layer,
(c) optionally applying a surface modification to the silicon oxide surface.

Preferred developments are specified in dependent claims 19 to 26.

The object of the invention is further achieved through the use of metallic effect pigment of any of claims 1 to 17 in cosmetics, plastics, and coating compositions, preferably inks, printing-inks, paints, and powder coating materials.

Lastly, the object of the invention is also achieved through provision of an article, where the article has and/or comprises metallic effect pigments of claims 1 to 17.

In the text below, the term “metallic effect pigments” refers only to those pigments whose metals are selected from the group consisting of aluminum, copper and alloys thereof, as indicated in claim 1, unless otherwise indicated.

According to one preferred embodiment, the alloy effect pigments are brass pigments which comprise zinc and copper and are also referred to as gold bronzes.

Brass effect pigments, typically referred to as “gold bronze”, preferably have a copper content of 70% to less than 100% by weight, preferably 75% to 90% by weight. The zinc content, accordingly, is preferably between 30% and 10% by weight, being for example 25% by weight, and there may optionally be up to 2% by weight, preferably below 1% by weight, of impurities of other metals.

In brass effect pigments or gold bronze effect pigments, the hue of the alloy is determined by the ratio of copper to zinc.

Gold bronze effect pigments are traded in characteristic natural shades, as “pale gold”, with a copper fraction of around 90% and a remainder of around 10% by weight of zinc; as “rich pale gold”, with a copper fraction of around 85% by weight and a remainder of around 15% by weight of zinc; and as “rich gold”, with a copper fraction of around 70% by weight and a remainder of around 30% by weight of zinc. The figure in % by weight here refers in each case to the total metal content of the metallic effect pigment, i.e., to the uncoated metallic effect pigment.

In one preferred embodiment the brass effect pigments include an “impurity” in the form of, for example, 0.1% to 2% by weight, preferably 0.5% to 1.8% by weight, of aluminum, based in each case on the total metal content of the uncoated metallic effect pigment. The alloys which have such a fraction of aluminum have proven more corrosion-stable as compared with brass effect pigments comprising exclusively copper and zinc.

According to one preferred variant of the invention, the metal cations present on the metallic effect pigment surface are at least partly different, preferably different, from the metal or metals present in the coated platelet-shaped metallic effect pigment.

According to one preferred variant of the invention, the metal cations present on the metallic effect pigment surface and/or in the silicon oxide layer (SiOₓ) are at least partly different, preferably different, from the metal or metals present in the platelet-shaped metallic effect pigment.

In accordance with the invention, the indication “on the metallic effect pigment surface” refers not only to the metallic effect pigment surface as such but also to a metallic effect pigment surface on which there is formed a metal oxide layer or on which a metal oxide layer has formed. Thus, for example, aluminum effect pigments have an aluminum oxide layer, or copper-containing effect pigments have a corresponding copper oxide-containing layer. The thickness of the metal oxide layer in these cases is typically within a range from 0.3 up to 20 nm—for example, from 1 nm to 10 nm. These metal oxide layers may be the natural metal oxide layers which always form as a result of contact of the metal surfaces with air under atmospheric conditions. In the case of copper-containing metallic effect pigments, however, a metal oxide layer may have been formed prior to the coating of the invention, by means, for example, of the well-known oxidization process. This produces “tarnish” colors, which, as a result of interference effects and the intrinsic coloring of the layer, lead to different hues in the gold-red range for these metallic effect pigments.

In the case of aluminum pigments, in addition to the very thin (layer thickness: around 3-5 nm) natural oxide layers, it is possible for further aluminum oxide, and/or aluminum hydroxide and/or aluminum oxide hydrate, to form during SiOₓ deposition, as a consequence of the sol-gel process, in which, indeed, water is present.

The inventors have surprisingly found that the stability of metallic effect pigments coated with silicon oxide SiOₓ with respect to corrosion is significantly enhanced if phosphorus-containing and/or sulfur-containing anions and metal cations are present independently of one another on the metallic effect pigment surface and/or in the silicon oxide layer (SiOₓ) in the proportions indicated above.

The silicon oxide SiOₓ is silicon oxide where x is a number from the range from 1 to 2. More preferably x is 1.8 to 2.0. Very preferably the silicon oxide is uncalcined silicon oxide, prepared by sol-gel processes, which may be termed SiO₂. Included here is the presence in the oxide of certain residual quantities of water and/or organic solvent, which may therefore also be present in a gellike state.

Calcined SiO₂ coatings, of the kind described in WO 2004/092284 A1, for example, are almost impossible to apply to metallic effect pigments. First of all, many of the commonplace metals, such as aluminum, for example, melt before the typical calcining temperatures are attained, and the metallic effect pigments are destroyed. In the case of aluminum pigments, furthermore, there is a safety risk in the initiation of a thermite reaction. Aluminum oxide (Al₂O₃) is known to be the most thermodynamically stable metal oxide, and the coating of an aluminum effect pigment with silicon oxide has a high potential to initiate a thermite reaction, owing to the high specific surface area of the metallic effect pigment and to the intimate contact between the reactants. The risk of activating this reaction by calcining (even below the melting point of aluminum) is too high to allow a safe production operation to be performed. Accordingly, the metallic effect pigments of the invention have an uncalcined silicon oxide layer.

Surprisingly, even small amounts of metal cations and phosphorus-containing and/or sulfur-containing anions on the metallic effect pigment surface and/or in the silicon oxide-containing layer are sufficient to increase the corrosion resistance of the metallic effect pigments.

The ratio of metal cations MC and phosphorus-containing anions P and/or sulfur-containing anions S, on the one hand, to silicon Si, on the other hand, is calculated on the basis of the atomic fractions of the elements of the metal of the metal cation and of phosphorus and/or sulfur, and related to the atomic fraction of the silicon present, in accordance with formula (I)

100%×(MC+P)/Si (I)

and/or in accordance with formula (II)

100%×(MC+S)/Si (II).

In accordance with the invention, the element ratios P and S, based in each case on the fraction of silicon from the silicon oxide layer, are in total, i.e., in summated form, in a range from 0.5% to 35%, more preferably from a total of 0.8% to 30%, even more preferably from a total of 1.0% to 20%. An element ratio of in total 0.8% to 18% has proven very suitable.

The amount of phosphorus and/or sulfur is preferably in a range from in total 1% to 15% and more preferably from in total 1.5% to 9%. If only phosphorus-containing or sulfate-containing anions are present, their respective element ratio may likewise be present in the proportions specified above, relative to the fraction of Si from the silicon oxide layer.

Surprisingly, the treatment of the metallic effect pigment surface with cations and with phosphorus- and/or sulfur-containing anions brings about an improvement in the stabilization of the metallic effect pigments toward corrosion, as compared with treatment of the metallic effect pigment surface with pure phosphoric acid or with organic phosphoric esters, i.e., without cations, such as triethyl phosphate, for example, with the subsequent application of a silicon oxide layer in each case. The reasons for this effect have to date not been understood.

Furthermore, surprisingly, a significant stabilization with respect to corrosion is also achieved when the cations and phosphorus- and/or sulfur-containing anions are present in the silicon oxide layer (SiOₓ layer).

According to a further variant of the invention, the metal cations and the phosphorus- and/or sulfur-containing anions are present predominantly in the SiOₓ layer. In this case it is further preferred for the concentration of the phosphorus- and/or sulfur-containing anions to be largely constant along the thickness of the SiOₓ layer.

The term “predominantly” in this context, and below, refers in accordance with the invention to more than 50%, preferably more than 59%, more preferably more than 66%. The term “predominantly” therefore also comprehends more than 76%, more preferably more than 86%, such than more than 96%, for example.

According to a further variant of the invention, the metal cations and the phosphorus- and/or sulfur-containing anions are present predominantly on the metallic effect pigment surface.

According to another variant of the invention, the metal cations with the phosphorus- and/or sulfur-containing anions are present at least partly with one another in the form of a sparingly soluble salt.

According to a further variant of the invention, the phosphorus- and/or sulfur-containing anions are present predominantly on the metallic effect pigment surface, and the metal cations are present predominantly in the SiOₓ layer.

According to a further variant of the invention, the metal cations are present predominantly on the metallic effect pigment surface, and the phosphorus- and/or sulfur-containing anions are present predominantly in the SiOₓ layer.

According to a further variant of the invention, the metal cations for the phosphorus-containing anions are selected from the group consisting of Ag(I), Cu(II), Cd(II), Cr(III), Co(II), Pb(II), Hg(I), Hg(II), Mg(II), Al(III), Zn(II), Sn(II), Ca(II), Sr(II), Ba(II), Mn(II), Bi(III), Zr(IV), Ni(II), Fe(II), Fe(III), and mixtures thereof, and also mixtures thereof with ammonium ions.

According to a further variant of the invention, the metal cations for sulfate anions as sulfur-containing anions are selected from the group consisting of Ag(I), Sb(III), Ca(II), Ba(II), Sr(II), Pb(II), Fe(III), and mixtures thereof. Particular preference is given here to Ba(II), Ca(II), and Fe(III), and very particular preference to Ba(II).

According to a further variant of the invention, the metal cations for sulfide anions as sulfur-containing anions are selected from the group consisting of Ag(I), Sb(III), Bi(III), Cd(II), Co(II), Cu(II), Ca(II), Ba(II), Pb(II), Mn(II), Ni(II), Sn(II), Sn(IV), Zn(II), Fe(II), and mixtures thereof. Particularly preferred are Ag(I), Cu(II), Fe(II), and Zn(II), and also mixtures thereof.

Especially preferred in this case are Zn(II) and Fe(II).

With very great advantage, the present invention permits the provision of metallic effect pigments which are stabilized with respect to corrosion and whose metals are selected from the group consisting of aluminum, copper, and alloys thereof, without having to accept any substantial detraction, preferably no detraction, from the optical properties of these metallic effect pigments, as compared with SiO₂ coated metallic effect pigments.

It is very surprising indeed that with such small proportions of phosphorus- and/or sulfur-containing anions and metal cations it has been possible to achieve an astonishing improvement in the gassing stability. The inventors do not as yet have any conclusive explanation for this.

At higher fractions of more than a total of 30%, the optical properties of the metallic effect pigments of the invention decrease significantly. Thus at higher levels, for example, “whitening” of the pigment is observed, leading to a reduced luster and a reduced light-dark flop.

Another effect of the small amounts of phosphorus- and/or sulfur-containing anions and metal cations is that they do not have any marked inherent color to thereby alter the optical properties of the metallic effect pigment.

The amount of silicon oxide, preferably SiO₂, is preferably in a range from 2% by weight to 25% by weight, preferably 3% by weight to 22% by weight, more preferably from 4% to 20% by weight, more preferably still from 5% by weight to 15% by weight, based in each case on the total weight of the metallic effect pigment substrate of the invention. In this context, the silicon oxide content is calculated as SiO₂.

The amount of silicon oxide preferably SiO₂, increases in the case of pigments with a high specific surface area. Metallic effect pigments with a high specific surface area are, more particularly, fine and thin pigments. Hence in the case of PVD aluminum pigments, for example, the SiO₂ content will tend to be in the upper range, and for conventional silver dollar pigments or cornflake pigments it will tend to take up a position in the lower quantity range. The amount of silicon oxide, preferably SiO₂, selected here is preferably not more than to achieve the desired gassing stability. If too much silicon oxide, preferably SiO₂, is applied, in other words more silicon oxide than is needed to maintain the gassing stability, all that happens is that the opacity and the optical properties as well are adversely affected, without any further advantage being obtained.

The phosphorus- and/or sulfur-based content is preferably in a range from 0.01% to 1.00% by weight, more preferably 0.02% to 0.50% by weight, and very preferably from 0.05% to 0.15% by weight, based in each case on the overall metallic effect pigment.

The amount of metal cations is preferably in a range from 0.03% to 1.4% by weight, more preferably 0.02% to 1.0% by weight, and very preferably from 0.05% to 0.5% by weight, based in each case on the overall metallic effect pigment.

The molar ratio of MC:P or of MC:S is preferably in a range from 10:1 to 1:10, preferably from 5:1 to 1:5, more preferably from 2:1 to 1:2. It is exceptionally preferred for the metal cations and the phosphorus-containing and/or sulfur-containing anions to be present in a stoichiometric ratio; this ratio may be, but need not be, equimolar.

According to one preferred development of the invention, the metal cations MC are selected such that they are able to form a sparingly soluble salt in each case with the phosphorus-containing and/or sulfur-containing anions in aqueous solution within a pH range of 5 to 8.

According to a further-preferred development of the invention, the metal cations are selected such that in water within a pH range from 5 to 8 and at a temperature of 25° C. they form sparingly soluble phosphates (PO₄³⁻) and/or hydrogenphosphates (HPO₄²⁻) and/or dihydrogenphosphates (H₂PO₄⁻) and/or sulfates (SO₄²⁻).

“Sparingly soluble” means in accordance with the invention, in the case of phosphorus-containing anions, more particularly of phosphates, that the corresponding solubility product of metal cation and phosphorus-containing anion, more particularly phosphate(s), has values, in a pH range from 5 to 8, of less than 10⁻¹⁰ (mol/l)^{m+n}, preferably less than 10⁻¹⁵ (mol/l)^{m+n}, more preferably less than 10⁻²⁰ (mol/l). Here, m is the stoichiometric coefficient of the metal cations, and n is the stoichiometric coefficient of the phosphorus-containing anions, more particularly phosphate ion(s).

In the case of sulfur-containing anions, more particularly in the case of sulfates, “sparingly soluble” means in accordance with the invention that the corresponding solubility product of metal cation and sulfur-containing anion, more particularly sulfate, has values of less than 10⁻⁵ (mol/l)^{m+k} and preferably less than 10⁻⁸ (mol/l)^{m+k}, where m is the stoichiometric coefficient of the metal cations and k is the stoichiometric coefficient of the sulfur-containing anions, more particularly of sulfate anions.

Preferred anions used are phosphorus-containing anions, more preferably phosphate ions.

According to one preferred development of the invention, in the case of phosphorus-containing anions, the metal cations are selected from the group consisting of Ag(I), Cu(II), Cd(II), Cr(III), Co(II), Pb(II), Hg(I), Hg(II), Mg(II), Al(III), Zn(II), Sn(II), Ca(II), Sr(II), Ba(II), Mn(II), Ni(II), Bi(III), Zr(IV), Fe(II), Fe(III), and mixtures thereof, and also mixtures thereof with ammonium ions.

According to one particularly preferred development of the invention, in the case of phosphorus-containing anions, the metal cations are selected from the group consisting of Ag(I), Cu(II), Mg(II), Al(III), Zn(II), Sn(II), Ca(II), Sr(II), Mn(II), Ni(II), Zr(IV), Fe(II), Fe(III), and mixtures thereof, and also mixtures thereof with ammonium ions. In the small amounts in which they are present in the metallic effect pigment of the invention, these metal cations have largely no toxicity and can therefore be used in more diverse applications.

According to one further-particularly preferred development of the invention, in the case of phosphorus-containing anions, the metal cations are selected from the group consisting of Mg(II), Al(III), Zn(II), Ca(II), Fe(II), Fe(III), and mixtures thereof.

Metal cations which have proven highly suitable in the case of phosphorus-containing anions are Fe(II), Fe(III), Al(III), Ca(II), and mixtures thereof. According to one very preferred development, Fe(II) cations are used. In this case the Fe(II) cations initially used may undergo at least partial oxidation to Fe(III) ions in the course of the reaction, as a result of oxygen present, for example.

According to one particularly preferred variant of the invention, the stoichiometric ratio of the metal cations and the phosphorus- and/or sulfur-containing anions is such that there is at least partial salt formation; in other words, such that the metal cations and the phosphorus- and/or sulfur-containing anions are present at least partly, and preferably largely completely, in the form of salts. This preferred variant applies for the variants where the metal cation and the phosphorus- and/or sulfur-containing anions are present predominantly both either in the SiOₓ layer or predominantly on the metallic effect pigment surface.

According to a further variant of the invention, at least 55% by weight, more preferably at least 70% by weight, more preferably still at least 92% by weight, of the metal cations and of the phosphorus-containing and/or sulfur-containing anions are present together as salt in the SiOₓ layer, preferably SiO₂ layer, and/or on the metallic effect pigment surface, the figures being based in each case on the total weight of metal cations and phosphorus-containing and/or sulfur-containing anions. It has proven very suitable for at least 95% by weight, furthermore at least 98% by weight, of the metal cations and of the phosphorus-containing and/or sulfur-containing anions to be present together in salt form, the figures being based in each case on the total weight of metal cations and phosphorus-containing and/or sulfur-containing anions. According to a further preferred embodiment of the invention, the metal cations and phosphorus-containing and/or sulfur-containing anions are present substantially completely, preferably completely, as salt, preferably as sparingly soluble salt in the SiOₓ layer, preferably SiO₂ layer, and/or on the metallic effect pigment surface.

The salts formed need not in this case be restricted solely to the metal cations preferred above, but may also be present in a mixture with, for example, ammonium ions—in the form of Mg(NH₄)³⁺, for example.

In the SiOₓ layer, preferably SiO₂ layer and/or on the metallic effect pigment surface, the sparingly soluble salts may be present in a largely molecular form or else in a colloidal form. They may be present, for example, largely as colloidal particles, preferably having dimensions in the range from on average 1 to 100 nm.

The phosphorus-containing anions are preferably phosphoric acid anions, phosphorous acid anions, phosphonic acid anions, phosphinic acid anions or salts thereof, i.e., phosphates, phosphites, phosphonates or phosphinites, respectively, or derivatives thereof. The species present may of course also be peroxo compounds and/or polyacid anions of the aforementioned acid anions or derivatives thereof. The derivatives are preferably esters, examples being alkyl esters, of the aforementioned acid anions. The alkyl esters of the aforementioned acid anions preferably have at least an alkyl chain length of C1 to C12, preferably C2 to C6.

Having proven very suitable are phosphoric anions, more particularly H₂PO₄⁻, H₂P₂O₇²⁻, HPO₄²⁻ and/or PO₄³⁻, or salts thereof, i.e., phosphates, or phosphorous acid anions, more particularly H₂PO₃⁻, HPO₃²⁻, H₂P₂O₅²⁻ and/or PO₃³⁻, or salts thereof, the phosphites. Furthermore, it is possible with preference, as phosphorus-containing anions, to use polyphosphates, metaphosphates, more particularly hexametaphosphate, or mixtures thereof.

PO₄³⁻ is used with exceptional preference as phosphoric acid anion.

Having proven to be a very suitable combination are Fe(II) and/or Fe(III) and/or Al(III) and/or Ca(II) ions and PO₄³⁻. An extremely suitable combination is Fe(II) and/or Fe(III) ions and PO₄³⁻.

The salts used during the preparation of the metallic effect pigments of the invention are preferably water-soluble, preferably slightly soluble, salts, more particularly alkali metal phosphates and/or alkali metal phosphites. Sodium salts and potassium salts of the aforementioned phosphorus-containing acid anions have proven very suitable.

Sulfur-containing anions used are preferably sulfates, sulfites, and sulfides, and also mixtures thereof.

Where the sulfur is present in the form of a sulfate, then preferred metal cations which are present with the sulfate anions in the SiOₓ layer, preferably SiO₂ layer, and/or on the metallic effect pigment surface are Ag(I), Sb(III), Ca(II), Ba(II), Sr(II), Pb(II), Fe(III) and mixtures thereof. Particularly preferred here are Ba(II), Ca(II), and Fe(III), and very preferably Ba(II).

Where the sulfur is in the form of a sulfide, then preferred metal cations which are present with sulfide anions in the SiOₓ layer, preferably SiO₂ layer, and/or on the metallic effect pigment surface are Ag(I), Sb(III), Bi(III), Cd(II), Co(II), Cu(II), Ca(II), Ba(II), Pb(II), Mn(II), Ni(II), Sn(II), Sn(IV), Zn(II), Fe(II), and mixtures thereof. Particularly preferred are Ag(I), Cu(II), Fe(II), and Zn(II), and also mixtures thereof. Especially preferred in this context are Zn(II) and Fe(II).

In one preferred embodiment, between the metal pigment surface and the SiOₓ layer, preferably SiO₂ layer, there may also be a layer of metal oxide, metal hydroxide and/or metal oxide hydrate. In a further-preferred embodiment in this case the metal cation is identical with that of the substrate material. The metal oxide layer is formed in this case preferably during the first phases of the deposition of SiOₓ, preferably of SiO₂.

The concentration of the metal cations and also of the phosphorus- and/or sulfur-containing anions may be determined by means of various methods. Thus, for example, it is possible to dissolve the coated metallic effect pigment in acid or base under defined conditions and to carry out an elemental analysis by means of ICP emission spectrometry (ICP: inductively coupled plasma). Here, the respective amount of the elements can be determined to a very high accuracy; however, it is not possible to determine the spatial position, within the layer construction, of the metallic effect pigments coated in accordance with the invention.

A further method of determining the concentrations is that of EDX analysis, as described in WO 2009/012995 A1, pages 26-29, hereby incorporated by reference.

This method as well provides information on the concentrations of the elements and can be confined three-dimensionally, by corresponding focusing, largely to the region of the SiOₓ layer, preferably the SiO₂ layer. A precise three-dimensional resolution, however, is not possible.

The third method lies in the detection of the relevant elements (Al, Si, O, P and/or S, MC) by means of ESCA measurements in combination with sputter profiles. In this case the concentration of the elements is obtained with locational resolution within the layer thickness of the SiO₂ layer and also, where appropriate, further layers. The method is again described in WO 2009/012995 A1, pages 25-26. As alternative or as a supplement it is likewise possible here to employ Auger spectroscopy in combination with sputter profiles.

It will be appreciated that the methods identified above may also be combined with one another in any way for the purpose of analyzing the samples.

Particularly preferred embodiments are the following metallic effect pigments coated in accordance with the invention:

A An aluminum effect pigment having an SiO₂ fraction of 5% to 15% by weight, calculated as SiO₂ and based on the weight of the metallic effect pigment, and phosphate and also Fe(II) and/or Fe(III) cations, these being present in each case independently of one another on the aluminum effect pigment surface and/or in the SiO₂ layer, and the element ratio in atomic fractions of Fe(II) and/or Fe(III) cations (MC) and phosphate P to silicon Si being defined in each case in accordance with the formula

100%×(MC+P)/Si (I)

and being situated in total in a range from 0.5% to 25%.

In more particularly preferred embodiments, the element ratio (MC+P)/Si is 0.8% to 15% and 1% to 8%.

In more preferred embodiments for A, the phosphate and the Fe(II) and/or Fe(III) cations are present predominantly as sparingly soluble salt in the SiO₂ layer.

In more preferred embodiments for A, the phosphate and the Fe(II) and/or Fe(III) cations are present predominantly as sparingly soluble salt on the aluminum effect pigment surface. This case includes the aluminum effect pigment surface also consisting of aluminum oxide and/or aluminum hydroxide and/or aluminum oxide hydrate with an average layer thickness of 5 to 20 nm, preferably of 8 to 15 nm, and the phosphate and the Fe(II) and/or Fe(III) cations being present predominantly in this layer and/or on this layer.

In more preferred embodiments for A, the phosphate is present predominantly on the aluminum effect pigment surface and the Fe(II) and/or Fe(III) cations are present predominantly in the SiO₂ layer. This case also includes the aluminum effect pigment surface also consisting of aluminum oxide and/or aluminum hydroxide and/or aluminum oxide hydrate with an average layer thickness of 5 to 20 nm, preferably of 8 to 15 nm, and the phosphate being present predominantly in this layer and/or on this layer.

B An aluminum effect pigment having an SiO₂ fraction of 5% to 15% by weight, calculated as SiO₂ and based on the weight of the metallic effect pigment, and phosphate and also Zn(II) cations, these being present in each case independently of one another on the aluminum effect pigment surface and/or in the SiO₂ layer, and the element ratio in atomic fractions of Zn(II) cations (MC) and phosphate P to silicon Si being defined in each case in accordance with the formula

100%×(MC+P)/Si (I)

and being situated in total in a range from 0.5% to 25%.

In more particularly preferred embodiments, the element ratio (MC+P)/Si is 0.8% to 15% and 1% to 8%.

In more preferred embodiments for B, the phosphate and the Fe(II) and/or Fe(III) cations are present predominantly as sparingly soluble salt in the SiO₂ layer.

In more preferred embodiments for B, the phosphate and the Zn(II) cations are present predominantly as sparingly soluble salt on the aluminum effect pigment surface. This case includes the aluminum effect pigment surface also consisting of aluminum oxide and/or aluminum hydroxide and/or aluminum oxide hydrate with an average layer thickness of 5 to 20 nm, preferably of 8 to 15 nm, and the phosphate and the Zn(II) cations being present predominantly in this layer and/or on this layer.

In more preferred embodiments for B, the phosphate is present predominantly on the aluminum effect pigment surface and the Zn(II) cations are present predominantly in the SiO₂ layer. This case also includes the aluminum effect pigment surface also consisting of aluminum oxide and/or aluminum hydroxide and/or aluminum oxide hydrate with an average layer thickness of 5 to 20 nm, preferably of 8 to 15 nm, and the phosphate being present predominantly in this layer and/or on this layer.

The metallic effect pigment, preferably aluminum effect pigment, coated using the method of the invention may optionally be provided with a surface modification adapted to the particular end use. For example, this surface modification may comprise silanes or may consist of silanes, and is applied preferably to the silicon oxide layer, preferably SiO₂ layer.

Examples of surface-modified aluminum effect pigments are described comprehensively in DE 198 20 112 A1, for example, the content of which is hereby incorporated by reference.

The metallic effect pigments have an average particle diameter which is preferably in the range from 1 μm to 200 μm, more preferably from 5 μm to 150 μm.

The metallic effect pigments used in accordance with the invention are platelet-shaped metallic pigments which can be obtained by milling atomized metal powder or by means of PVD techniques (PVD: physical vapor deposition).

The metallic effect pigments are preferably aluminum and/or copper or brass (gold bronze) effect pigments, and more preferably aluminum effect pigments.

The aluminum effect pigments may be of the “cornflake” type or of the “silver dollar” type.

Particularly advantageous is the use of aluminum effect pigments in accordance with the disclosure content of DE 103 157 15 A1 and DE 10 2006 062271, the content of each of which is hereby incorporated by reference. Aluminum effect pigments such as these, also identified as “Platindollar®” or “Silvershine® S”, are produced by wet grinding and in terms of their pigment properties such as average thickness and thickness distribution are virtually comparable with PVD aluminum effect pigments.

In contrast to PVD aluminum effect pigments, these “Platindollar” or “Silvershine” aluminum effect pigments, obtained by wet grinding, do not have an absolutely planar surface, as is the case with PVD aluminum effect pigments. PVD aluminum effect pigments, furthermore, have relatively straight fracture edges, whereas the aluminum effect pigments obtained by wet grinding have an irregularly shaped marginal region which may also be referred to as a frayed marginal region.

With particular advantage, metallic effect pigments, preferably aluminum effect pigments, having an average thickness h₅₀, as determined via thickness counting by scanning electron microscopy, of 15 to below 100 nm and a relative breadth of the thickness distribution Δh, as determined via thickness counting by scanning electron microscopy, and which is calculated on the basis of the corresponding cumulative frequency curve of the relative frequencies, according to the formula Δ=100×(h₉₀−h₁₀)/h₅₀, of 30% to 140% can be used, as disclosed in DE10315715 A1 and DE 102006062271.

The cumulative frequency curve is also referred to as the cumulative undersize curve.

A further-preferred embodiment relates to PVD metallic effect pigments, preferably PVD aluminum effect pigments, which can be produced by the method of the invention to provide metallic effect pigments coated in accordance with the invention.

The metallic effect pigments of the invention have an outstanding stability toward corrosion, obviating the need to apply further protective layers. According to one preferred variant of the invention, the metallic effect pigments of the invention have no further organic and/or inorganic protective layer. A surface modification is not understood to be an organic protective layer.

According to a further preferred embodiment, the metallic effect pigments of the invention have no further layer, and so the silicon layer, preferably SiO₂ layer, is the outermost layer.

Further provided with the present invention is an entirety of metallic effect pigments, coated in accordance with the invention, which comprises at least three populations of metallic effect pigments of the invention, whose d₅₀ diameters each differ by 2 to 6 μm, the smallest d₅₀ of a metallic effect pigment from the entirety being not more than 5 μm. An entirety of this kind is able to generate a wide spectrum of different optical impressions on the part of the metallic effect pigments, in respect of luster, sparkle, lightness flop etc.

In one further-preferred embodiment, the entirety comprises at least four populations of metallic effect pigments of the invention whose d₅₀ diameters each differ by 3 to 5 the smallest d₅₀ of a metallic effect pigment from the entirety being not more than 5 μm.

The entirety of the metallic effect pigments coated in accordance with the invention may of course also comprise more populations of metallic effect pigments of the invention, having different d₅₀ values and different breadths of the size distributions, this being dependent on the requirements for a desired optical appearance.

The method of the invention for producing the metallic effect pigments of the invention comprises the following steps:
(a) applying silicon oxide to the metallic effect pigments, with reaction of alkoxysilane(s) and/or silicon halide(s) in organic solvent with water in the presence of an acid and/or base as catalyst,
(b) adding phosphorus- and/or sulfur-containing anions and metal cations to the metallic effect pigments before or during step (a), with phosphorus- and/or sulfur-containing anions and metal cations being taken up onto the metallic effect pigment surface and/or into the silicon oxide layer,
(c) optionally applying a surface modification to the silicon oxide surface.

According to one preferred variant of the invention, the method for producing silicon oxide-coated metallic effect pigments comprises the following steps:
(a) applying silicon oxide to the metallic effect pigments, with reaction of alkoxysilane(s) and/or silicon halide(s) in organic solvent with water in the presence of an acid and/or base as catalyst,
(b) adding phosphorus- and/or sulfur-containing anions and metal cations to the metallic effect pigments before or during step (a), with phosphorus- and/or sulfur-containing anions and metal cations being taken up predominantly into the silicon oxide layer,
(c) optionally applying a surface modification to the silicon oxide surface.

With the method variant above, metallic effect pigments of the invention are provided in which the phosphorus- and/or sulfur-containing anions and metal cations are present predominantly in the silicon oxide layer, preferably SiO₂.

According to a further preferred variant of the invention, the method for producing silicon oxide-coated metallic effect pigments comprises the following steps:
(a) adding phosphorus- and/or sulfur-containing anions and metal cations to the metallic effect pigments, with phosphorus- and/or sulfur-containing anions and metal cations being taken up predominantly onto the metallic effect pigment surface,
(b) applying silicon oxide to the metallic effect pigments treated according to step (a), with reaction of alkoxylsilane(s) and/or silicon halide(s) in organic solvent with water in the presence of an acid and/or base as catalyst,
(c) optionally applying a surface modification to the silicon oxide surface.

With the above method variant, metallic effect pigments of the invention are provided in which the phosphorus- and/or sulfur-containing anions and metal cations are present predominantly on the metallic effect pigment surface.

According to a further preferred variant of the invention, the method for producing silicon oxide-coated metallic effect pigments comprises the following steps:
(a) applying silicon oxide to the metallic effect pigments, with reaction of alkoxysilane(s) and/or silicon halide(s) in organic solvent with water in the presence of an acid and/or base as catalyst,
(b) adding phosphorus- and/or sulfur-containing anions and/or metal cations to the metallic effect pigments before or during step (a), with phosphorus- and/or sulfur-containing anions being taken up predominantly onto the metal surface, and with metal cations being taken up predominantly into the silicon oxide layer,
(c) optionally applying a surface modification to the silicon oxide surface.

With the above method variant, metallic effect pigments of the invention are provided in which the phosphorus- and/or sulfur-containing anions are present predominantly on the metallic effect pigment surface and the metal cations are present predominantly in the silicon oxide layer, preferably SiO₂.

The uptake of the metal cations and/or of the phosphorus- and/or sulfur-containing anions, respectively, onto the metallic effect pigment surface and/or into the silicon oxide layer may be controlled, for example, by the sequence in which the metal cations and/or the phosphorus- and/or sulfur-containing anions, respectively, are added. It is also possible, optionally, to utilize the different kinetics of the respective chemical reactions, as for example the kinetics of formation of a sparingly soluble salt, in order to control the uptake of the metal cations and/or of the phosphorus- and/or sulfur-containing anions, respectively, onto the metallic effect pigment surface and/or into the silicon oxide layer.

According to a further preferred variant of the invention, the method for producing silicon oxide-coated metallic effect pigments comprises the following steps:
(a) applying silicon oxide to the metallic effect pigments, with reaction of alkoxysilane(s) and/or silicon halide(s) in organic solvent with water in the presence of an acid and/or base as catalyst,
(b) adding phosphorus- and/or sulfur-containing anions and/or metal cations to the metallic effect pigments before or during step (a), with metal cations being taken up predominantly onto the metal surface, and with phosphorus- and/or sulfur-containing anions being taken up predominantly into the silicon oxide layer,
(c) optionally applying a surface modification to the silicon oxide surface.

With the method variant above, metallic effect pigments of the invention are provided in which the metal cations are present predominantly on the metallic effect pigment surface, and the phosphorus- and/or sulfur-containing anions are present predominantly in the silicon oxide layer, preferably SiO₂.

In the method of the invention, metallic effect pigments are coated with silicon oxide, this section of the method comprising the following steps:
a) applying silicon oxide to the metallic effect pigments, with reaction of alkoxysilane(s) and/or silicon halide(s) in organic solvent with water in the presence of an acid and/or base as catalyst,
b) adding phosphorus- and/or sulfur-containing anions and metal cations to the metallic effect pigments before or during step (a), with phosphorus- and/or sulfur-containing anions and metal cations being taken up into the silicon oxide layer,
c) optionally applying a surface modification to the silicon oxide surface.

The method according to step (a) is also referred to as a sol-gel process. It can be catalyzed with addition of acid and/or a base.

In one preferred variant, in accordance with the invention, of this section of the method, the reaction according to step (a)

(i) is carried out in a first step with addition of acid and in a second step with addition of base, or
(ii) the reaction is carried out in a first step with addition of base and in a second step with addition of acid.

Surprisingly, the improved gassing stability and also, in particular, the advantageous opacity properties of the metallic effect pigments of the invention have been further improved by means of this two-stage method for applying silicon oxide, preferably SiO₂.

The silicon oxide is preferably SiO₂.

According to one particularly preferred development of the invention, the phosphorus-containing anions and the metal cations are added in separate solutions to the metallic effect pigments to be treated, preferably with stirring. The metallic effect pigments to be treated are present preferably in the form of a dispersion, preferably in an organic or aqueous organic solvent. The addition of the phosphorus- and/or sulfur-containing anions on the one hand and of the metal cations on the other hand may take place in separate steps simultaneously or in succession. Depending on the preferred variant of the method of the invention, first the solution comprising the phosphorus- and/or sulfur-containing anions, and subsequently the solution comprising the metal cations, are added to the metallic effect pigments, or vice versa.

The metal cations and the phosphorus- and/or sulfur-containing anions are preferably added in the form of slightly soluble salt compounds or acids. Thus, for example, Fe(II) ions can be added as FeSO₄ salts, and phosphates as hydrogenphosphates or dihydrogen-phosphates of sodium or of potassium.

It is particularly preferred in accordance with the invention, therefore, if the metal cations and the phosphorus- and/or sulfur-containing anions are able to form sparingly soluble salts only in the reaction mixture.

The solution with the phosphorus- and/or sulfur-containing anions and the solution with the metal cations may also be added in portions to the metallic effect pigments. In the case of alternating addition, the metallic effect pigments are preferably contacted first with the solution which comprises the phosphorus-containing anions.

The time of commencement of the addition of the solution with the phosphorus- and/or sulfur-containing anions and/or of the solution with the metal cations to the reaction mixture is preferably situated within a time period ranging from 1 hour before the start of the sol-gel reaction to 4 hours after the start of the sol-gel reaction which brings about the deposition of silicon oxide, preferably of SiO₂, on the metallic effect pigment surface. The start of reaction is identified as the point in time at which all of the major reaction partners in the sol-gel reaction (metallic effect pigments, alkoxysilane, water, and catalyst) are present in the reaction mixture and, preferably, the temperature is elevated.

With further preference, the point in time of the commencement of the addition of the solution with the phosphorus- and/or sulfur-containing anions and/or of the solution with the metal cations to the reaction mixture is situated within a range from 30 minutes before the start to 3 hours after the start, and more preferably from 0.0 h to 2 h after the start, of the sol-gel reaction.

The point in time of the addition of the metal cations and/or of the phosphorus- and/or sulfur-containing anions is also influenced substantially by the method variant selected.

In the case of predominant uptake of the metal cations and of the phosphorus- and/or sulfur-containing anions into the silicon oxide layer, it is preferred first to start the sol-gel reaction for applying the silicon oxide layer and then to add the metal cations and the phosphorus- and/or sulfur-containing anions.

In the case of predominant uptake of the metal cations and of the phosphorus- and/or sulfur-containing anions onto the metallic effect pigment surface, it is preferred first to add the metal cations and the phosphorus- and/or sulfur-containing anions and then to start the sol-gel reaction for applying the silicon oxide layer.

In the case of predominant uptake of the metal cations onto the metallic effect pigment surface and of predominant uptake of the phosphorus- and/or sulfur-containing anions into the silicon oxide layer, it is preferred first to add the metal cations and subsequently to start the sol-gel reaction for applying the silicon oxide layer, and then to add the phosphorus- and/or sulfur-containing anions.

In the case of predominant uptake of the phosphorus- and/or sulfur-containing anions onto the metallic effect pigment surface and of predominant uptake of the metal cations into the silicon oxide layer, it is preferred first to add the phosphorus- and/or sulfur-containing anions and subsequently to start the sol-gel reaction for applying the silicon oxide layer, and then to add the metal cations.

As already observed above, the uptake of the metal cations and/or of the phosphorus- and/or sulfur-containing anions, respectively, onto the metallic effect pigment surface and/or into the silicon oxide layer, respectively, may also be influenced via the different reaction kinetics.

Preferably, therefore, depending on the method variant, before, during or after the addition of the solution(s) with the phosphorus- and/or sulfur-containing anions, and/or of the solution with the metal cations, the silicon oxide layer, preferably SiO₂ layer, is applied with hydrolysis of alkoxysilane(s) and/or silicon halide(s).

The alkoxysilane(s) and/or the silicon halide(s) here are or is hydrolyzed in the organic solvent or solvent mixture by the existing water and/or by added water. As a result of the hydrolysis, OH groups are formed on the silicon atoms, and are also referred to as silanol groups. The silanol groups undergo condensation, with elimination of water, to form an Si—O—Si network. This Si—O—Si network then precipitates in the form of a sol/gel onto the metallic effect pigments, thereby encapsulating them or enveloping them with silicon oxide, preferably SiO₂.

The solutions with the phosphorus- and/or sulfur-containing anions and the solution with the metal cations are added, as observed above, before, during and/or after the start of the sol-gel reaction.

Preferred phosphorus-containing anions are phosphate ions. Preferred sulfur-containing anions are sulfate ions.

The inventors have discovered that, surprisingly, metallic effect pigments in particular that have been treated with phosphorus- and/or sulfur-containing anions and with metal cations, as elucidated above, and have also been coated by an at least two-stage method with silicon oxide, preferably SiO₂, exhibit improved performance properties. The two-stage method for applying silicon oxide, preferably SiO₂, is based here on different catalysts, and encompasses an acid-catalyzed step and a base-catalyzed step.

The basis for this surprising effect has not hitherto become clear. The pH governs a change in the ratio of the rate of hydrolysis of alkoxy group(s) of the alkoxysilanes and/or of the halide(s) of the silicon halides to silanol group(s) to the rate of the condensation of the silanol groups with another, with formation of Si—O—Si bonds.

In one preferred variant of the invention, the hydrolysis of the alkoxy group(s) takes place primarily with addition of acid(s). In the case of this method variant, the pH is preferably in a range from pH 3 to 7, preferably from pH 4 to 6.5. A pH value range from pH 4.5 to pH 6 is also very suitable.

According to one preferred development of this method variant, the condensation of the generated silanol groups to form Si—O—Si bonds takes place primarily with addition of base. The pH in this case is preferably in a pH value range from more than pH 7 to pH 11, more preferably from pH 7.5 to pH 10. A pH in the range from pH 8 to pH 9.5 has also proven very suitable.

According to one variant of the invention, it is also possible to apply a continuous pH gradient during the coating operation. In this case, preferably, the pH is changed continuously from acidic to basic, by continuous addition of the corresponding reagents.

In a further-preferred embodiment, there is a pH discontinuity between the step of hydrolysis with addition of acid(s) and the step of condensation with addition of base(s). The difference in pH between the first and second steps is situated preferably in a range from 0.3 to 4 pH units, more preferably from 0.5 to 3 pH units, and more preferably still from 0.7 to 2 pH units.

In this very much preferred embodiment of the method of the invention, the reaction takes place with acid catalysis in a first step and with basic catalysis in a second step. The reaction scheme (III) here is as follows:

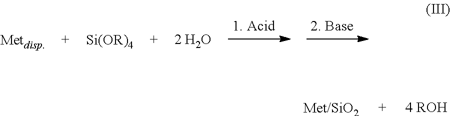

The acid(s) and/or base(s) each act catalytically, by influencing the reaction rate of the hydrolysis to silanol groups and/or condensation of the silanol groups to form Si—O—Si bonds of a silicon oxide network, preferably a silicon dioxide network. Kinetically preferred in this case, in the first step, is the hydrolysis of the alkoxysilanes to silanols (IIIa), and in the step the condensation of the silanols (IIIb).

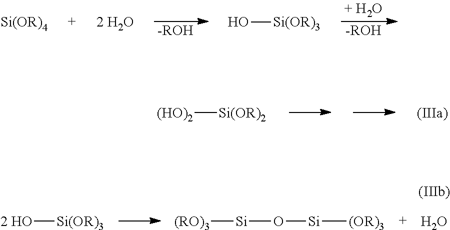

In the case of acid catalysis, sol-gel processes are known to first yield linear and/or cyclic and/or ladder-like siloxane oligomers which have only a low silanol group fraction. The reason lies in the decreasing hydrolysis rate of the oligomeric alkoxysilanes as compared with the rate of hydrolysis of the monomeric alkoxysilanes.

With basic catalysis, in contrast, the formation of three-dimensional siloxane oligomer structures with a high silanol content is preferred. In this case there is an increasing hydrolysis rate of the oligomers relative to the rate of hydrolysis of the monomeric alkoxysilanes, and also a high condensation rate.

In the case of rapid condensation of silanol groups to form Si—O—Si bonds, deleteriously, metallic effect pigments with a small particle diameter are deposited or precipitated, together with the rapidly forming silicon oxide, typically SiO₂, on the surface of metallic effect pigments having a larger particle diameter. The metallic effect pigments with smaller particle diameter are therefore encapsulated in the silicon oxide envelope of the metallic effect pigments with larger particle diameter.

This has twin disadvantages:

For good opacity (hiding power), i.e., the area of substrate surface hidden per unit weight of pigment, an essential factor is the fraction of metallic effect pigments having a small particle diameter, also referred to as fine fraction, in a metallic effect pigment preparation.

A metallic effect pigment is normally present in a particle size distribution. As the breadth of the particle size distribution goes up, there is an increase in the opacity of the metallic effect pigment. The fine fraction of a metallic effect pigment, preferably aluminum effect pigment, is characterized for example by the D₁₀ of the cumulative distribution of the size distribution curve. The size distribution curve is typically determined by means of laser granulometry.

As a result of the reduction in the fine fraction of a metallic effect pigment preparation by the precipi-tation thereof together with the nascent silicon oxide onto the metallic effect pigments with larger particle diameter, the opacity of the silicon oxide-coated metallic effect pigment preparation is reduced.

The second disadvantage is that the metallic luster of the metallic effect pigment preparation coated with silicon oxide is reduced. Owing to the precipitation of the fine fraction onto the metallic effect pigments with the larger pigment diameter, incident light is scattered to an increased extent quite simply because of the increased edge fraction. This effect is particularly deleterious to the luster of the metallic effect pigments.

In the case of this first variant of the method of the invention, the precipitation of silicon oxide onto the metallic effect pigments is slow, presumably because of the slow generation of the silanol groups. Presumably, in light of the slower precipitation of the silicon oxide, the fine fraction of the metallic effect pigment preparation is not entrained, but is instead separately coated with silicon oxide, with, consequently, no adverse effect on the opacity and the metallic luster.

In the second step of the method, after the metallic effect pigments have been coated with a first layer of silicon oxide, preferably SiO₂, a second layer of silicon oxide, preferably SiO₂, is subsequently applied with addition of base.

Surprisingly, after the addition of base in the second step, there is no substantial agglomeration, and preferably no agglomeration, of metallic effect pigments with small particle diameter at or on metallic effect pigments with larger particle diameter. It is presumed that this effect can be attributed to the coverage of the metallic effect pigment surface with the first silicon oxide coating.

It is surprising, furthermore, that in the two stage silicon oxide application method used in accordance with the invention, which may also be referred to as a sol-gel process, metallic effect pigments are coated with the same amounts of silicon oxide, preferably SiO₂, as in a purely basic procedure used typically in the prior art, despite the fact that the reaction begins in an acidic or acidified reaction solution and ends, preferably by way of a pH gradient, in a less acidic, neutral or basic, range.

This is true in particular in light of the fact that from the prior art it is evident that the precipitation of silicon dioxide onto an aluminum effect pigment surface with catalysis with a base results in better layer formation and a better yield (e.g., EP 1 619 222 A1, EP 1 953 195 A1), which is why the prior art in principle uses a basic catalyst (A. Kiehl, K. Greiwe, Progr. in org. Coatings 37 (1999), 179-183).

In accordance with the invention it is possible, surprisingly, to operate in a pH range from pH 4 to 7, which was explicitly ruled out in the prior art, such as in EP 1 953 195 A1, for example, when carrying out an operation conducted exclusively in the acidic pH range.

Surprisingly, therefore, it has been found that the metallic effect pigments coated by the two-stage sol-gel method in accordance with the invention with silicon oxide, preferably SiO₂, have an opacity which is improved relative to that of metallic effect pigments coated with silicon oxide by the conventional sol-gel process.

In the case of the second method variant, in accordance with reaction scheme (IV), the basic catalysis takes place in a first step and the acidic catalysis subsequently, in a second step:

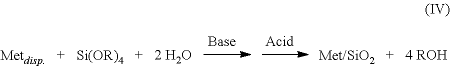

Surprisingly, this method route as well leads to enhanced opacity on the part of the metallic effect pigments of the invention.

In one preferred embodiment, following the addition of the basic catalyst, the acidic catalyst used in the second step is added rapidly. This means that, based on the point in time at which the basic catalyst is added, the period for addition is preferably from 15 min to 4 h, more preferably 20 min to 2.5 h, and more preferably still 30 min to 1.5 h. In one embodiment of the second method variant, the pH may be changed continuously from basic to acidic by addition of acid. In the case of another preferred embodiment of the second method variant, the addition of acid leads to a pH discontinuity. The difference in pH between the first and second steps is situated preferably within a range from 0.3 to 4 pH units, more preferably from 0.5 to 3 pH units, and more preferably still from 0.7 to 2 pH units.

For both method variants, the acids and/or bases used as catalysts are in principle the same.

As acids it is possible to use organic and/or inorganic acids. Organic acids are particularly preferred.

The organic acid(s) used as acidic catalyst in accordance with the invention comprises preferably 1 to C atoms, more preferably 1 to 6 C atoms, and very preferably 1 to 4 C atoms.

The organic radical of these acids may comprise linear, cyclic or branched alkyl, alkenyl, aryl, and aralkyl radicals.

The acids may be monobasic, dibasic or tribasic acids, with monobasic or dibasic acids being particularly preferred.

Above 8 C atoms, the acid strength is generally too low and the steric shielding is too high to allow use as an effective catalyst.

In accordance with one preferred variant, the organic acid used as acidic catalyst is selected from the group consisting of formic acid, acetic acid, propionic acid, oxalic acid, malonic acid, maleic acid, succinic acid, anhydrides of the stated acids, and mixtures thereof. It is especially preferred to use formic acid, acetic acid or oxalic acid and also mixtures thereof.

In accordance with a further variant of the invention, the inorganic acid used as acidic catalyst in accordance with the invention is preferably selected from the group consisting of nitric acid, sulfuric acid, phosphoric acid, hydrochloric acid, boric acid, hydrofluoric acid, and mixtures thereof. In this case it is preferred to use nitric acid and/or hydrofluoric acid.

Using phosphoric and/or sulfuric acid is a preferred embodiment, providing at the same time the preferred anions, phosphate and/or sulfate.

The basic catalyst is preferably an organic base and more preferably an amine. The amines in question may be primary, secondary or tertiary.

In a further-preferred embodiment, the amine has 1 to 8, more preferably 1 to 6, and very preferably 1 to 5 C atoms. Amines having more than 8 C atoms often have an excessive steric bulk to allow them to be used as effective catalysts.

According to one preferred variant of the invention, the amine is selected from the group consisting of dimethylethanolamine (DMEA), monoethanolamine, diethanolamine, triethanolamine, ethylenediamine (EDA), tert-butylamine, monomethylamine, dimethylamine, trimethylamine, monoethylamine, diethylamine, triethylamine, pyridine, pyridine derivative, aniline, aniline derivative, choline, choline derivative, urea, urea derivative, hydrazine derivative, and mixtures thereof.

As basic aminic catalyst it is particularly preferred to use ethylenediamine, monoethylamine, diethylamine, monomethylamine, dimethylamine, trimethylamine, tri-ethylamine or mixtures thereof.

Additionally preferred as basic catalyst is an aminosilane selected preferably from the group consisting of 3-aminopropyltriethoxysilane (AMEO), 3-aminopropyltrimethoxysilane (AMMO), N-2-aminoethyl-3-aminopropyltriethoxysilane (DAMEO), N-2-aminoethyl-3-aminopropyltriemthoxysilane (DAMO), N-2-aminoethyl-3-aminomethylpropyltriethoxysilane, triamino-functional trimethoxysilane (Silquest A-1130), bis(gamma-tri-methoxysilylpropyl)amine (Silquest A-1170), N-ethyl-gamma-aminoisobutyltrimethoxysilane (Silquest A-Link 15), N-phenyl-gamma-aminopropyltrimethoxysilane (Silquest Y-9669), 4-amino-3,3-dimethylbutyltrimethoxy-silane (Silquest Y-11637), N-cyclohexylaminomethyl-methyldiethoxysilane (GENIOSIL XL 924), (N-cyclohexyl-aminomethyl)triethoxysilane (GENIOSIL XL 926), (N-phenylaminomethyl)trimethoxysilane (GENIOSIL XL 973), and mixtures thereof.

As basic catalyst it is particularly preferred to use 3-aminopropyltriethoxysilane (AMEO), 3-aminopropyl-trimethoxysilane (AMMO), N-2-aminoethyl-3-aminopropyl-triethoxysilane (DAMEO), N-2-aminoethyl-3-aminopropyl-triemthoxysilane (DAMO) or mixtures thereof.

As basic catalyst it is of course also possible to use a mixture of at least one amine and at least one amino-silane.

The inorganic base is preferably selected from the group consisting of ammonia, hydrazine, sodium hydroxide, potassium hydroxide, ammonium hydroxide, ammonium carbonate, ammonium hydrogencarbonate, sodium carbonate, sodium hydrogencarbonate, potassium carbonate, potassium hydrogencarbonate, and mixtures thereof.

It is particularly preferred in this context to use ammonia and/or hydrazine, and especially preferred to use ammonia.

The silicon oxide is preferably SiO₂. SiO₂ produced by sol-gel processes is known to be amorphous. It has a significant fraction of bound water. This water may be intercalated into the SiO₂. Furthermore, the silicon oxide can contain a fraction of unhydrolyzed alkoxy groups.

For producing silicon oxide, preferably SiO₂, is preferred to use alkoxysilanes.

The alkoxysilane used in accordance with the invention preferably comprises di-, tri- and/or tetraalkoxy-silanes. Tetraalkoxysilane is especially preferred. When tetraalkoxysilane is used, the hydrolysis results in formation of four silanol groups, which, with condensation, produce a high degree of crosslinking, i.e., a silicon oxide coating, preferably SiO₂ coating, having a good barrier effect. When di- or trialkoxy-silanes are used, hydrolysis, accordingly, produces two or three silanol groups, which are able to condense to form an Si—O—Si network. The use of di- or trialkoxy-silanes permits the introduction of organic groups, as for example of alkyl groups, or polymers into the silicon oxide coating, to form an inorganic-organic hybrid layer. The di- or trialkoxysilanes can also be dubbed organosiloxanes.

An alkoxysilane in accordance with the invention is any monomeric or polymeric silicon compound having at least one alkoxy group. Tetraalkoxysilane used advantageously comprises tetramethoxysilane, tetraethoxysilane, tetra-isopropoxysilane, and condensates thereof, or mixtures of these.

It is particularly advantageous to use, as tetraalkoxy-silane, tetraethoxysilane and/or oligomers of tetra-ethoxysilane.

When using alkoxysilane(s), preferably tetra-alkoxysilane(s), the great advantage is that no salts are produced. This is advantageous both environmentally and in regard of possible agglomeration processes during the sol-gel reaction, since salts disrupt the electrostatic stabilization of the pigment particles.

Another advantage is that, in contrast to the use of silicon halides, there is no release of halides such as chloride ions, for example. Halide ions, as is known, may promote the corrosion of metals.

The silicon halide used in accordance with the invention preferably comprises di-, tri- and/or tetra-silicon halides. Silicon tetrahalide is especially preferred. The above remarks relating to di-, tri- and/or tetraalkoxysilanes apply correspondingly.

A silicon halide in accordance with the invention is any monomeric or polymeric silicon compound having at least one halide group.

Silicon halides used are preferably tetrasilicon halides. Tetrasilicon halides used are preferably tetrasilicon fluoride, tetrasilicon chloride, tetra-silicon bromide, tetrasilicon iodide or mixtures thereof, or mixed halides of these compounds.

For coating with silicon oxide, preferably SiO₂, the metallic effect pigments are dispersed in the organic solvent with stirring, the reaction mixture is brought preferably to elevated temperature, and preferably water is added. Subsequently, the first catalyst, i.e., acid or base, depending on method variant, and also, preferably, alkoxysilane are added, and, after a first reaction time, the second catalyst, i.e., base or acid, depending on method variant, is added. The mixture is left under these conditions for a second reaction time.

It is then possible, optionally, for surface modifiers to be added. The mixture is cooled preferably to room temperature and the coated metallic effect pigments are largely separated from the solvent, to form a metallic effect pigment filtercake.

A substantial disadvantage of the conventionally employed basic sol-gel coating of a metallic effect pigment is the lack of or inadequacy of balance in the performance properties of the resultant coated metallic effect pigments, especially with regard to hiding power and corrosion stability, in an application medium, such as in a pigmented waterborne paint, for example.

As is known, inadequate corrosion stability on the part of a metallic effect pigment, more particularly an aluminum effect pigment, in an aqueous application medium leads to reaction with the water, with hydrogen being evolved and the metallic effect pigment breaking up. Even slight corrosion impairs the optical properties, more particularly the specular gloss.

The opacity or hiding power of a pigmented medium means the capacity of the pigmented medium to hide the color or color differences in a substrate (DIN 55987).

For the optical appearance of a metallic effect pigment, more particularly of an aluminum effect pigment, one important optical assessment criterion for pigmented applications is the particle size of the pigment and its distribution, known as the particle size distribution.

The hiding power or opacity which characterizes the optical appearance of a metallic effect pigment preparation, more particularly an aluminum effect pigment preparation, increases as the breadth of the particle size distribution goes up, since in that case the amount of the fine fraction contained is increasingly greater. Generally speaking, the hiding power or opacity increases as the fineness of the metallic effect pigment in the metallic effect pigment preparation goes up.

The metallic effect pigments preferably have a particle size distribution with a D₅₀ of 2 to 75 μm, more preferably of 2 to 30 μm, and particularly preferably of 2.5 to 20 μm, and very preferably of 2.5 to 12 μm.

The finer the metallic effect pigments, the greater the loss of opacity in comparison to the uncoated starting material, in the case of the conventional products. Surprisingly, the metallic effect pigments of the invention exhibit an improvement in the opacity particularly in the case of these relatively fine pigments.

The metallic effect pigment used as starting pigment in the method of the invention is preferably dispersed into a solvent mixture which comprises alkoxysilane, preferably tetraalkoxysilane, this mixture being composed of, or including, organic solvent and optionally water. The acidic catalyst, preferably organic or inorganic acid(s), is added preferably after the dispersing of the metallic effect pigment in the organic solvent and optional heating of the dispersion to reaction temperature. The water required for the hydrolysis may already be present in the organic solvent or may be added at a later point in time.

Then organic or inorganic base(s) is/are introduced as basic catalyst into the reaction mixture comprising metallic effect pigments, alkoxysilanes, preferably tetraalkoxysilanes, water, and acid(s), in order to start the second stage of the method of the invention.

Organic solvents used are preferably alcohols, glycols, esters, ketones, and mixtures of these solvents. Particularly preferred is the use of alcohols or glycols or mixtures thereof, and especially preferred is the use of alcohols.

As the alcohol it is advantageous to use methanol, ethanol, isopropanol, N-propanol, T-butanol, N-butanol, isobutyl alcohol, pentanol, hexanol or mixtures thereof.

Particular preference is given to using ethanol and/or isopropanol.

As glycol, it is advantageous to use butylglycol, propylglycol, ethylene glycol or mixtures thereof.

For coating with silicon oxide, preferably SiO₂, the reaction mixture present is reacted preferably at a temperature within a range from 20° C. up to the boiling point of the respective solvent or solvent mixture. With particular preference the reaction temperature is within a range from 50° C. up to a temperature which is preferably 5° C. below the boiling point of the respective solvent or solvent mixture. A preferred reaction temperature range for coating with silicon oxide, preferably SiO₂, is the temperature range extending from 75° C. to 82° C.

The reaction time, for the first and/or second stage of in coating with silicon oxide, preferably SiO₂, in the method of the invention, is situated preferably, in each case, within a range of 2 to 20 h, more preferably 3 to 8 hours.

The metallic effect pigment, preferably aluminum effect pigment, coated in accordance with the invention and optionally surface-modified is separated from the reaction mixture and can then be passed on to its intended use. For example, the metallic effect pigment of the invention can be processed further as a powder or paste and can then be introduced into inks, printing-inks, paints, plastics, cosmetics, etc.

This invention additionally provides, furthermore, a metallic effect pigment of the invention which is in the form of a powder, dry product or paste, and is distinguished by the fact that in and/or on the silicon oxide layer, preferably SiO₂ layer, and/or in the solvent of the paste there is

0.01%-1% by weight of organic and/or inorganic acid and
0.01%-1% by weight of organic and/or inorganic base, where the % by weight figures are based on the total weight of the pigment.

The dry product of the invention may take the form, for example, of granules, pellets, sausages, tablets, briquettes, etc. The dry product may take the form of a low-dust or dust-free metallic effect pigment preparation. The residual moisture content here may be in a range from 0.5% up to 29% by weight, preferably up to 1% to 24% by weight, more preferably from 3% up to 14% by weight, more preferably still from 4% up to 9% by weight, with these figures being based in each case on the total weight of the dry product. The dry product preferably further comprises binder, generally organic polymer(s) and/or resin(s), and also, optionally, additive(s). The amount of binder in the dry product is preferably in a range from 0.5% to 20% by weight and more preferably from 1% to 5% by weight, based in each case on the total weight of the dry product.

The acids and/or bases here may be at least partly in ionic form, as for example in the form of a salt. The bases may also be at least partly in the form of a salt with the acidic silanol groups (Si—OH).

These features are an inevitable consequence of the two-stage method for applying silicon oxide, preferably SiO₂. With particular preference, the concentrations of the organic and/or inorganic acid and of the organic and/or inorganic base independently of one another are 0.015%-0.5% by weight and very preferably 0.015%-0.2% by weight, based on the total weight of the pigment. In view of the two-stage method for applying silicon oxide, not only acid and/or acid anions but also bases are located in and/or on the silicon oxide layer, preferably SiO₂ layer. These components are catalyst residues which are adsorbed and/or enclosed in the silicon oxide layer, preferably SiO₂ layer.

The acids and bases are preferably located pre-dominantly in the silicon oxide layer, preferably SiO₂ layer.

If the metallic effect pigment of the invention is in the form of a paste, the acids and/or bases may also be largely in the solvent of this paste. Thus the solvent of the paste may also leach the acids and/or bases which to start with are predominantly in the silicon oxide layer, preferably SiO₂ layer, from that layer. This may be the case in particular after a certain storage time of the metallic effect pigment of the invention, with the subsequent surface adsorption of the acid and/or base onto the silicon oxide layer, preferably SiO₂ layer, being possible.

A paste in the context of this invention is a mixture comprising the metallic effect pigment of the invention and a solvent, with the amount of metallic effect pigment being preferably 5% to 80% by weight and the amount of metallic effect pigment and the solvent being preferably at least 95% by weight, based on the paste.

Depending on the nature, and more particularly on the specific surface area and also the surface properties, of the metallic effect pigment of the invention, and possibly on the nature of the solvent, the preparation may take the form of a dry preparation or a paste.

The amount of metallic effect pigment in the paste is heavily dependent on its specific surface area. If the desire is to bring very thin metallic effect pigments having average thicknesses below 100 nm, such as PVD pigments, for example, into a pasty form, then a very high solvent fraction is necessary for this purpose. Accordingly, the amount of such pigments is preferably 5% to 30% by weight and more preferably 10% to 20% by weight, based on the total weight of the paste.

Pastes such as these should always be viewed as a precursor product for the subsequent application of the metallic effect pigment.

For thicker metallic effect pigments having average thicknesses>100 nm, metallic pigment contents of above 20% to 80% by weight, preferably 30% to 75% by weight, and more preferably 50% to 70% by weight, based in each case on the total weight of the paste, are generally sufficient.

The paste may further comprise additional constituents such as additives, for example. The fraction of further components, however, is low, since this is not an end application (formulation). The amount of metallic effect pigment and the solvent in the paste is therefore preferably at least 97% by weight and more preferably at least 98% by weight, based in each case on the total weight of the paste.

The solvents present in the paste are preferably solvents familiar in the paints and printing-ink industry. Since the principal end use of the metallic effect pigments of the invention is as water-based paints or printing-inks, particularly preferred pastes are those in which the solvent comprises or consists of water. The water fraction of the paste of the invention is preferably 20% to 100% by weight, more preferably 30% to 90% by weight, and very preferably 40% to 80% by weight, based on the weight of the solvent in the paste. Pastes such as these are particularly preferred for environmental reasons, on account of their low VOC fraction.

The residues of acid and base that are present in the silicon oxide layer, preferably SiO₂ layer, amount in general to not more than 1% by weight in each case. This can be attributed to the fact that the reaction, as described above, takes place in a mixture of organic solvent and water. Generally speaking, the major fraction of the catalysts used as acid and/or base is dissolved in this solvent mixture. The fraction of catalyst included in the pigment therefore corresponds only to a small fraction of the catalyst employed overall.

The organic acid and/or anions thereof does not comprehend long-chain fatty acids, i.e., saturated or unsaturated fatty acids having 12 to 30 C atoms or having 14 to 22 C atoms. Such fatty acids are used as lubricants during the grinding of metallic pigments. Consequently, as a result of the production process, any metallic effect pigment produced by grinding will contain these fatty acids. During the silicon oxide, preferably SiO₂, coating operation, the fatty acids bound to the metal surface are largely detached. In certain fractions, however, they may be incorporated into the silicon oxide layer, preferably SiO₂ layer, or may be adsorbed on the pigment surface after the end of the coating procedure.

Because of their low acid strength and because of the high steric shielding, however, these long-chain fatty acids are not used as catalysts in sol-gel methods for producing silicon oxide layers, preferably SiO₂ layers.

Accordingly, in accordance with the invention, the acids and bases which are present in and/or on the silicon oxide layer, preferably SiO₂ layer, in the stated proportions are understood to include only those which are used in sol-gel processes as a catalyst for the deposition of silicon oxide.

Preferred acids or bases are those compounds already stated above.

In the case of aminosilanes as basic catalyst, it should be noted that these are commonly also used as surface modifiers, in order to allow effective attachment of the metallic effect pigment to the binder. For that purpose, however, it is common to use amounts of at least 1% by weight, based on the metallic effect pigment.

The analytical detection of the bases and/or acids is made preferably by means of gas chromatography and mass spectroscopy. In this case, in a preferred way, the coated metallic effect pigment is taken up in a suitable organic solvent, treated in an ultrasound bath at room temperature or slightly elevated temperature for at least 15 minutes, and admixed with—for example—hexadecane as internal standard. The solid is removed by centrifugation and the supernatant solution is used as the injection solution for the gas chromatograph. The supernatant solution may also, optionally, be concentrated in an appropriate way, if the concentration of the acid or base to be detected is otherwise too low.

Where the acids and/or bases to be detected are not known, then qualitative detection takes place preferably by means of GC/MS. In this case it is preferred to use a medium-polarity column. These columns, named DB5, are packed typically with 5% diphenal and 95% dimethylpolysiloxane.

Where the substances to be detected are known, then their quantitative determination can take place by means of gas chromatography. In this case it is preferred to use an apolar column. Calibration takes place in a customary way known to the skilled person. These columns, named DB1, are typically packed with 100% dimethylpolysiloxane. A detection system suitable is a flame ionization detector (FID). The exact parameters for setting the gas chromatograph (e.g., column length and width, column pressure, etc.) are known to the skilled person.

The gas chromatograph used is preferably a GC/FID Autosystem XL (from Perkin Elmer).

The acids and bases may also be determined by means of other mass-spectrometric techniques, such as TOF-SIMS, for example. In this case, continuous erosive sputter of the sample may be necessary, in order to allow detection of the residual quantities of catalyst present in the silicon oxide layer, preferably SiO₂ layer as well. The method can be applied preferably where aminosilanes are used as basic catalyst, since these aminosilanes are naturally bonded covalently to the silicon oxide layer, preferably SiO₂ layer and do not have to be leached from it by extraction.

The metallic effect pigments of the invention find use in cosmetics, plastics, and coating compositions, preferably inks, printing-inks, paints or powder coating materials. Particularly preferred in this context are waterborne paints, aqueous printing-inks or cosmetics.

The metallic effect pigments of the invention are incorporated into their respective application media in a customary way. An article may then be coated with these application media thus pigmented. Said article may be, for example, a vehicle body, an architectural facing element, etc.

In the case of plastics, the metallic pigment of the invention may also be incorporated for coloring into the application medium in the mass. The articles have and/or comprise the metallic effect pigments of the invention.

The examples below and FIG. 1 illustrate the invention in more detail, though without restricting the invention:

### FIGURES

FIG. 1 shows the hiding power (opacity) of the aluminum effect pigments coated in accordance with inventive examples 1 to 3 and comparative examples 1 to 3, and also in comparison to the aluminum effect pigment coated in accordance with the teaching of WO 03/01/014228 A1 of Merck, Darmstadt, Germany.

### INVENTIVE EXAMPLE 1

150 g of a commercial aluminum effect pigment paste of the series STAPA METALLUX 3580 from Eckart GmbH, Hartenstein, Germany, having a size distribution of d₁₀=7.5 μm, d₅₀=12.1 μm, and d₉₀=18.7 μm and a solids content of 60% by weight, based on the total weight of the paste, were dispersed at room temperature in 500 ml of isopropanol. In the course of 30 minutes, in each case independently of one another, 0.54 g of phosphoric acid (85% strength; Merck, Darmstadt, Germany) in solution in 15 g of water, and 2.25 g of iron(II) sulfate heptahydrate in solution in 15 g of water, were added continuously to the pigment dispersion. After 30 minutes, 45 g of tetraethoxysilane and 0.4 g of oxalic acid in solution in 15 g of water were added, and the mixture was conditioned at 78° C. After a stirring time of 4 hours without further addition, the batch was stirred for a further 4 hours, and every 2 hours during this a solution of 1.5 g of ethylenediamine in 13 g of isopropanol was added. After a further hour and after the end of the sol-gel reaction, for organic-chemical aftercoating, 1.3 g of Dynasylan AMMO, available from Degussa AG, Rheinfelden, Germany, were added, and the batch was stirred for 15 minutes. The reaction mixture was subsequently cooled to room temperature and filtered with suction through a Büchner funnel.

The product obtained had a size distribution of d₁₀=8.1 μm, d₅₀=13.0 μm, d₉₀=19.3 μm.

Elemental ratios in atom percent of the pigment coating, determined by EDX (12 KV):

### Si: 80.5%

### P: 6.6%

### Fe 12.9%

### Quantitative Determination of the Amount of Silicon, Phosphorus, and Iron by Means of EDX

The elemental composition of the pigment coating can be determined by various methods. The elemental coating is preferably determined by means of EDX analysis (energy dispersive X-ray analysis). This is carried out here using an instrument in which an electron microscope is integrated, an example being the EDAX Genisis, version 3.60, from EDAX.

The analytical procedure is elucidated below:

In the EDX analysis method, the imaging electron beam of the electron microscope, dependent on its energy and on the material, penetrates a distance into the sample surface and delivers its energy to the atoms located there. Owing to the high energy of the beam electrons, electrons are ejected from the near-nucleus shell (K or L shell) of the excited atoms. This operation gives rise to x rays by a twofold mechanism. The sharp braking of the electrons generates a continuously distributed x-radiation, the bremsstrahlung, and the refilling of the shells generates a discrete x-ray spectrum, the characteristic linear spectrum of the atom. These linear spectra allow the elements to be identified unambiguously.

The x-radiation spectrum emitted by the sample under analysis is measured by means of an energy-dispersive x-ray spectrometer. The spectrum is made up of the bremsstrahlung background and a series of x-ray spectral lines. The position of the lines allows the emitting elements to be determined; the height of the lines is a measure of their relative amounts in the sample.

In EDX elemental analysis, there are a number of boundary conditions to be observed for correct measurement of the element amounts. The samples for analysis must be

a) homogeneous in their composition,
b) sufficiently thick for the imaging electron beam to be absorbed completely within the sample, and
c) freely accessible to the electron beam, without disruptive effects of a matrix and/or a substrate.

The higher the atomic number of the elements, the stronger the bonding of the near-nucleus electrons. Consequently, the ionization energy required increases in line with the atomic number. The kinetic energy of the electron beam must be adapted to the elements to be analyzed. The depth of penetration of the electron beam into the material under analysis, however, is dependent on its energy. The electron beam penetrates the sample in an intensity distribution which has a pear-shaped structure and is also referred to as a pear-shaped excitation cloud. When thin layers are being analyzed, it must be borne in mind that they are easily punctured by high-energy electrons. If thin layers (range below 250 nm) are to be measured, the kinetic energy must only amount to a few KeV. In the case of heavier elements, therefore, the excitation of the higher shells is implemented instead. Analysis then takes place via evaluation of the L or M lines of the elements.

Specifically, the procedure for analyzing thin-layer, platelet-shaped pigments is as follows:

Prior to the analysis, the EDX measuring unit is calibrated using suitable, commercially available standards (from ASTIMEX).

By means of electron-microscopy imaging, the layer thickness of the layer under investigation must be ascertained. Elemental analysis with a relatively high voltage (approximately 10 to 20 kV) provides information on all of the elements present in the sample under analysis, and also on further elements located in the underlying substrate. From the thickness and from the elemental composition of the layer, a Monte Carlo simulation (program: EDAX Flight-E, version 3.1-E, from EDAX International) determines the electron energy at which the layer volume is fully filled by the penetrating electron beam, but still not punctured. In that case the pear-shaped excitation cloud has the greatest volume.

In the next step a determination is made as to whether and, if so, which x-ray lines are excited for this radiation energy. The kinetic excitation energy is adapted if appropriate to the spectral lines.

A first sample measurement with the parameters thus determined is carried out and analyzed. If x-ray lines of substrate elements are seen in the spectrum, then the radiation energy setting is too high, and is corrected.

Then a number of measurements are carried out on the layer, with the radiation voltage increasing in steps, and are evaluated. There should be only minor fluctuations in the element amounts found. If the fraction of light elements in the analysis begins to show a marked fall as the voltage increases, the radiation energy is too high, and is reduced.

With the optimum parameters determined in this way, measurements are carried out on a number of locations on the layer, and the element amounts are ascertained.

Quantitative Determination of Residues of Carboxylic Acid and/or Amine:

Carboxylic acid is determined by means of gas chromatography with an internal standard. For this purpose, a sample of the coated metallic effect pigment paste was taken up with a defined amount of acetone in the case of the carboxylic acid determination and taken up in a defined amount of ethanol in the case of the amine determination, then treated in an ultrasound bath for 15 minutes, and admixed with hexadecane as internal standard. The solid was removed by centrifugation and the supernatant solution was used as the injection solution for the gas chromatograph (GC/FID Autosystem XL (Perkin Elmer)). The carboxylic acid content was analyzed with the following outline parameters:

### Column: 30 m OV 101 0.53 mm

Temperature program: 45° C. 1 min isothermal 5° C./min 180° C.
Injection board: 250° C.

### Detector: 320° C.

### Quantitative Determination of EDA:

The sample was prepared in the same way as indicated above. The gas chromatograph (GC/FID Autosystem XL (Perkin Elmer)) was equipped with the following outline parameters:

### Column: 30 m OV 101 0.53 mm

Temperature program: 75° C. 10° C./min 200° C.
Injection board: 250° C.

### Detector: 320° C.

In each case 0.01% by weight of oxalic acid and 0.02% of EDA were detected, based on the weight of the coated aluminum pigment.

### INVENTIVE EXAMPLE 2

261 g of a commercial aluminum effect pigment paste of the series STAPA METALLUX 2156 from Eckart GmbH, Hartenstein, Germany, having a size distribution of d₁₀=9 μm, d₅₀=17 μm, and d₉₀=28 μm and a solids content of 70% by weight, based on the total weight of the paste, were dispersed at room temperature in 300 ml of isopropanol. Over the course of 30 minutes, in each case independently of one another, 0.54 g of phosphoric acid (85% strength; Merck, Darmstadt, Germany) in solution in 15 g of water, and 2.25 g of iron(II) sulfate heptahydrate in solution in 15 g of water, were added continuously to the pigment dispersion. After 30 minutes, 45 g of tetraethoxysilane and 0.4 g of oxalic acid in solution in 15 g of water were added, and the mixture was conditioned at 78° C. After a stirring time of 4 hours without further addition, the batch was stirred for 4 hours more, during which every 2 hours a solution of 1.5 g of ethylenediamine in 13 g of isopropanol was added. After a further hour and after the end of the sol-gel reaction, for organic-chemical aftercoating, 1.11 g of Dynasylan AMMO, available from Degussa AG, Rheinfelden, Germany, were added, and the batch was stirred for 15 minutes. It was subsequently cooled to room temperature and filtered with suction on a Büchner funnel. The product obtained had a size distribution of d₁₀=9.6 μm, d₅₀=17.5 and d₉₀=27.9 μm.

Elemental ratios in atom percent of the pigment coating, determined by EDX (12 KV), as described in example 1:

### Si: 82.5%

### P: 6.0%

### Fe 11.5%

The amount of oxalic acid and EDA in the pigment was determined by the method described in example 1. 0.01% by weight of oxalic acid and 0.02% by weight of EDA were found, based on the weight of the coated aluminum pigment.

### INVENTIVE EXAMPLE 3

250 g of a commercial aluminum effect pigment paste of the series STAPA METALLUX 8154 from Eckart GmbH, Hartenstein, Germany, having a size distribution of d₁₀=9 μm, d₅₀=20 μm, and d₉₀=32 μm and a solids content of 65% by weight, based on the total weight of the paste, were dispersed at room temperature in 600 ml of isopropanol. Over the course of 30 minutes, in each case independently of one another, 0.54 g of phosphoric acid (85% strength; Merck, Darmstadt, Germany) in solution in 15 g of water, and 2.25 g of iron(II) sulfate heptahydrate in solution in 15 g of water, were added continuously to the pigment dispersion. After 30 minutes, 45 g of tetraethoxysilane and 0.4 g of acetic acid in solution in 15 g of water were added, and the mixture was conditioned at 78° C. After a stirring time of 4 hours without further addition, the batch was stirred for 4 hours more, during which every 2 hours a solution of 1.5 g of ethylenediamine in 13 g of isopropanol was added. After a further hour and after the end of the sol-gel reaction, for organic-chemical aftercoating, 1.2 g of Dynasylan AMMO, available from Degussa AG, Rheinfelden, Germany, were added, and the batch was stirred for 15 minutes. It was subsequently cooled to room temperature and filtered with suction on a Büchner funnel.

The product obtained had a size distribution of d₁₀=9.6 μm, d₅₀=20.1 and d₉₀=33.1 μm.

Elemental ratios of the pigment coating, determined by EDX (12 KV), as described in example 1:

### Si: 82.5%

### P: 5.5%

### Fe: 12.0%

The amount of acetic acid and EDA in the pigment was determined by the method described in example 1. 0.01% by weight of oxalic acid and 0.02% by weight of EDA were found, based on the weight of the coated aluminum pigment.

### INVENTIVE EXAMPLE 4

300 g of a commercial aluminum effect pigment dispersion of the series Metalure W-52012 IL from Avery Dennison, Schererville, Ind., USA, having a solids content of 20% by weight, based on the total weight of the paste, were dispersed at room temperature in 800 ml of isopropanol. Over the course of 30 minutes, in each case independently of one another, 0.24 g of phosphoric acid (85% strength; Merck, Darmstadt, Germany) in solution in 10 g of water, and 0.84 g of iron(II) sulfate heptahydrate in solution in 10 g of water, were added continuously to the pigment dispersion. After 30 minutes, 45 g of tetraethoxysilane and 3 g of ethylenediamine in solution in 40 g of water were added, with heating to 78° C. and further conditioning at this temperature. After two hours, a solution of 3 g of ethylenediamine in 30 g of isopropanol is added. This is repeated after 3 hours more, whereupon the batch is stirred at 78° C. for a further 4 hours. After the end of the sol-gel reaction, for organic-chemical aftercoating, 3.0 g of Dynasylan AMMO, available from Degussa AG, Rheinfelden, Germany, were added, and the batch was stirred for 15 minutes. It was subsequently cooled to room temperature and filtered with suction on a Büchner funnel.

Gassing test (for the description see below):

30 g/0 ml (d: day(s))

Elemental ratios of the pigment coating, determined by EDX (12 KV), as described in example 1:

### Si: 91.0%

### P: 3.7%

### Fe: 5.3%

The amount of EDA in the pigment was determined by the method described in example 1. 0.04% by weight of EDA were found, based on the weight of the coated aluminum pigment.

### INVENTIVE EXAMPLE 5

150 g of a commercial aluminum effect pigment paste of the series STAPA METALLUX 3580 from Eckart GmbH, Hartenstein, Germany, having a size distribution of d₁₀=7.5 μm, d₅₀=12.1 μm, and d₉₀=18.7 μm and a solids content of 60% by weight, based on the total weight of the paste, were dispersed at room temperature in 500 ml of isopropanol. After an hour, 5.10 g of barium acetate (from Merck, Darmstadt, Germany) in solution in 15 g of water were added continuously over the course of an hour to the pigment dispersion. After 30 minutes, 45 g of tetraethoxysilane and 2.00 g of concentrated sulfuric acid in solution in 15 g of water were added, and the mixture was conditioned at 78° C. After a stirring time of 4 hours without further addition, the batch was stirred for 4 hours more, during which every 2 hours a solution of 2.0 g of ethylenediamine in 13 g of isopropanol was added. After a further hour and after the end of the sol-gel reaction, for organic-chemical aftercoating, 1.3 g of Dynasylan AMMO, available from Degussa AG, Rheinfelden, Germany, were added, and the batch was stirred for 15 minutes. It was subsequently cooled to room temperature and filtered with suction on a Büchner funnel.

The product obtained had a size distribution of d₁₀=8.5 μm, d₅₀=14.1 μm, and d₉₀=19.6 μm.

Elemental ratios of the pigment coating, determined by EDX (12 KV):

### Si: 81.8%

### S: 7.1%

### Ba: 11.1%

The amount of EDA residues in the pigment was determined by the method described above. 0.01% by weight of EDA was found, based on the weight of the coated aluminum pigment.

### INVENTIVE EXAMPLE 6

150 g of a commercial aluminum effect pigment paste of the series STAPA METALLUX 3580 from Eckart GmbH, Hartenstein, Germany, having a size distribution of d₁₀=7.5 μm, d₅₀=12.1 μm, and d₉₀=18.7 μm and a solids content of 60% by weight, based on the total weight of the paste, were dispersed at room temperature in 500 ml of isopropanol. After two hours, in each case independently of one another, 0.54 g of phosphoric acid (85% strength; Merck, Darmstadt, Germany) in solution in 15 g of water, and 2.17 g of zinc sulfate heptahydrate in solution in 15 g of water, were introduced continuously into the pigment dispersion, simultaneously, over the course of one hour. After 30 minutes, 45 g of tetraethoxysilane and 0.4 g of oxalic acid in solution in 15 g of water were added, and the mixture was conditioned at 78° C. After a stirring time of 4 hours without further addition, the batch was stirred for 4 hours more, during which every 2 hours a solution of 1.5 g of ethylenediamine in 13 g of isopropanol was added. After a further hour and after the end of the sol-gel reaction, for organic-chemical aftercoating, 1.3 g of Dynasylan AMMO, available from Degussa AG, Rheinfelden, Germany, were added, and the batch was stirred for 15 minutes. It was subsequently cooled to room temperature and filtered with suction on a Büchner funnel.

The product obtained had a size distribution of d₁₀=8.2 μm, d₅₀=13.5 μm, and d₉₀=19.6 μm.

Elemental ratios of the pigment coating, determined by EDX (12 KV):

### Si: 83.5%

### P: 5.6%

### Zn: 10.9%

The amount of oxalic acid and EDA residues in the pigment was determined by the method described above. 0.01% by weight of oxalic acid and 0.01% by weight of EDA was found, based on the weight of the coated aluminum pigment.

### COMPARATIVE EXAMPLE 1

150 g of the STAPA METALLUX 3580 aluminum effect pigment paste used in inventive example 1 were dispersed in 500 ml of isopropanol at room temperature. After 30 minutes, 45 g of tetraethoxysilane were added and the mixture was heated to 78° C. and conditioned at that temperature. Then a solution of 1.8 g of ethylenediamine (EDA) in 45 g of water was added and the reaction mixture was stirred for three hours. Subsequently a solution of 1.7 g of ethylenediamine with 18.6 g of isopropanol was added to the reaction mixture, which was stirred for seven hours. After the end of the sol-gel reaction, for organic-chemical aftercoating, 1.2 g of Dynasylan AMMO, available from Degussa AG, Rheinfelden, Germany, were added. The metallic effect pigment mixture obtained was cooled to room temperature and filtered with suction on a Büchner funnel.

The product obtained had a size distribution of d₁₀=10.3 μm, d₅₀=15.6 μm, and d₉₀=22.4 μm.

The amount of EDA in the pigment was determined by the method described in example 1. 0.02% by weight of EDA was found, based on the weight of the coated aluminum pigment.

### COMPARATIVE EXAMPLE 2

150 g of the STAPA METALLUX 2156 aluminum effect pigment paste used in inventive example 2 were dispersed in 350 ml of isopropanol at room temperature. After 30 minutes, 45 g of tetraethoxysilane were added and the mixture was heated to 78° C. and conditioned at that temperature. Then a solution of 1.7 g of ethylenediamine in 45 g of water was added and the reaction mixture was stirred for seven hours. Subsequently a solution of 1.7 g of ethylenediamine with 18.6 g of isopropanol was added to the reaction mixture, which was stirred for 3 hours. After the end of the sol-gel reaction, for organic-chemical aftercoating, 1.2 g of Dynasylan AMMO, available from Degussa AG, Rheinfelden, Germany, were added. The metallic effect pigment mixture obtained was cooled to room temperature and filtered with suction on a Büchner funnel.

The product obtained had a size distribution of d₁₀=12.8 μm, d₅₀=20.6 μm, and d₉₀=31.4 μm.

The amount of EDA in the pigment was determined by the method described in example 1. 0.02% by weight of EDA was found, based on the weight of the coated aluminum pigment.

### COMPARATIVE EXAMPLE 3

250 g of the STAPA METALLUX 8154 aluminum effect pigment paste used in inventive example 3 were dispersed in 600 ml of isopropanol at room temperature. After 30 minutes, 45 g of tetraethoxysilane were added and the mixture was heated to 78° C. and conditioned at that temperature. Then a solution of 2.0 g of ethylenediamine in 60 g of water was added and the reaction mixture was stirred for seven hours. Subsequently a solution of 2.0 g of ethylenediamine with 39 g of isopropanol was added to the reaction mixture, which was stirred for three hours. After the end of the sol-gel reaction, for organic-chemical aftercoating, 1.2 g of Dynasylan AMMO, available from Degussa AG, Rheinfelden, Germany, were added. The metallic effect pigment mixture obtained was cooled to room temperature and filtered with suction on a Büchner funnel.

The product obtained had a size distribution of d₁₀=10.3 μm, d₅₀=20.2 μm, and d₉₀=33.0 μm.

The amount of EDA in the pigment was determined by the method described in example 1. 0.02% by weight of EDA was found, based on the weight of the coated aluminum pigment.

### COMPARATIVE EXAMPLE 4

### In Accordance with WO 03/014228 A1

83.3 g of STAPA METALLUX 3580 were predispersed in 500 ml of acetone in a glass beaker for 15 minutes. Then 1 g of phosphoric acid (85% strength) was added. The mixture was stirred at room temperature for 30 minutes. After the end of the time, the solids in the batch were filtered off with suction on a Büchner funnel and washed with 300 g of acetone. The resulting solid was taken up in 850 g of ethanol and admixed with 197.5 g of water and 22.4 g of ammonia solution (25% strength in water). The resulting solution was then heated to 65° C. and admixed with 35.2 g of tetraethoxysilane in 76 ml of ethanol. After the end of the sol-gel reaction (7 h), for organic-chemical aftercoating, 3.0 g of Dynasylan AMMO, available from Degussa AG, Rheinfelden, Germany, were added. The solution was stirred for a further 13 hours, after which the pigment was filtered off with suction on a Büchner funnel.

The product obtained had a size distribution of d₁₀=13.5 μm, d₅₀=31.0 μm, d₉₀=61.8 μm.

### COMPARATIVE EXAMPLE 5

### PVD

300 g of a commercial aluminum effect pigment paste of the series Metalure W-52012 IL from Avery Dennison, Schererville, Ind., USA, having a solids content of 20% by weight, based on the total weight of the paste, were dispersed in 800 ml of isopropanol at room temperature. After a dispersing time of 30 minutes, 45 g of tetraethoxysilane and 3.0 g of ethylenediamine in 40 g of water were added, with the mixture being heated to 78° C. and conditioned at that temperature thereafter. After three hours, a solution of 3.0 g of ethylenediamine in 30 g of isopropanol was added. This was repeated after three hours more, whereupon the batch was stirred at 78° C. for a further 4 hours. After the end of the sol-gel reaction, for organic-chemical aftercoating, 3.0 g of Dynasylan AMMO, available from Degussa AG, Rheinfelden, Germany, were added, and the batch was stirred for 15 minutes. It was then cooled to room temperature and filtered with suction on a Büchner funnel.

The amount of EDA in the pigment was determined by the method described in example 1. 0.03% by weight of EDA was found, based on the weight of the coated aluminum pigment.

### Gassing Test:

All of the coated metallic effect pigments were subjected to two gassing tests. For gassing test 1, 8.6 g of coated Al pigment in the form of a paste were incorporated into 315 g of colorless waterborne mixing varnish (ZW42-1100, BASF Würzburg) and brought to a pH of 8.2 using dimethanolethanolamine. 300 g of this paint were introduced into a gas wash bottle, which was closed with a double-chamber gas bubble counter. The volume of gas produced was read off on the basis of the water volume displaced in the lower chamber of the gas bubble counter. The gas wash bottle was conditioned at 40° C. in a water bath and the test was carried out over a maximum of 30 days. The test is passed if no more than 10.5 ml of hydrogen has been evolved after 30 days.

A further gassing test, test 2, for determining the corrosion resistance of metallic effect pigments, involves blending with iron oxide (red iron oxide gassing test, BASF). For this purpose a pasting is prepared from 9.75 g of aluminum (calculated from the solids content of the paste), 19.5 g of red iron oxide tinting paste, 19.5 g of butyl glycol, and 15 g of binder. 23 g of this pasting are introduced into a varnish mixture (from BASF, Würzburg) and adjusted to a pH of 8.0. 300 g of the resulting paint are introduced into a gas wash bottle and closed with a double-chamber gas bubble counter. The resulting gas volume was read off on the basis of the displaced water volume in the lower chamber of the gas bubble counter. The gas wash bottle was conditioned at 40° C. in a water bath and the test was carried out over a maximum of 56 days. The test is passed if no more than 5 ml of hydrogen has been evolved after 56 days.

Table 1 gives the particle size distributions of the starting pigments and of the coated pigments, and the resistance of the pigments in the gassing test, for the pigments produced in inventive examples 1 to 6 and in comparative examples 1 to 4.

### Opacity:

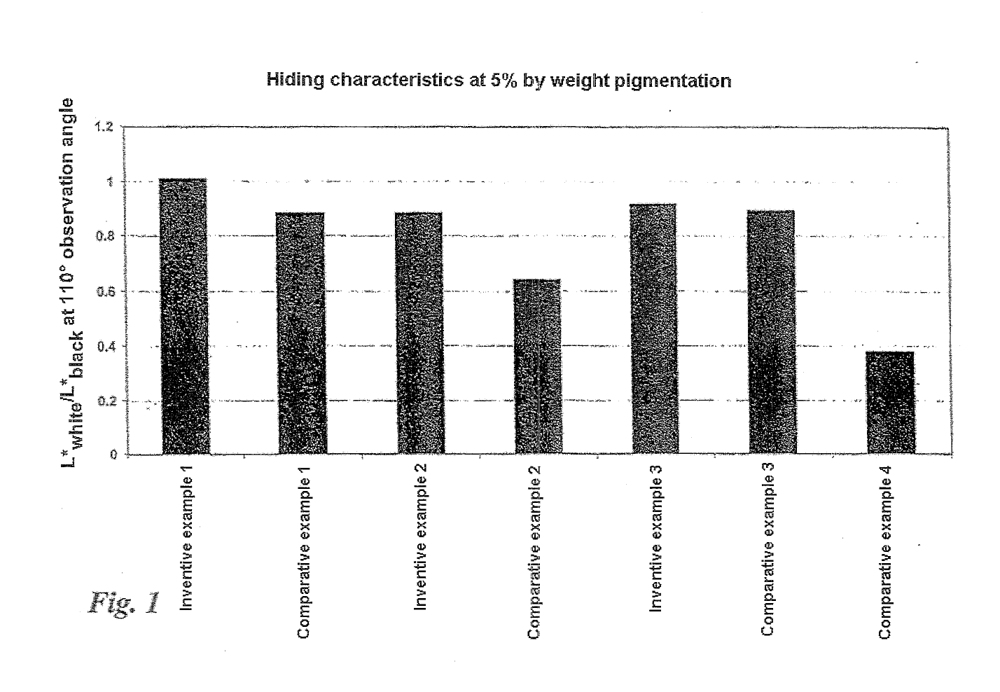

In order to evaluate the hiding power (opacity) of the aluminum effect pigments of the inventive and comparative examples, knife drawdowns thereof (pigmentation in each case 5% by weight of coated metallic effect pigment in Erco Bronzemischlack RE 2615 Farblos [colorless bronze mixing varnish], wet film thickness: 50 μm) were produced on a commercial black/white opacity chart (type 24/5, 250 cm³, Erichsen GmbH & Co KG, Hemer-Sundwig), and then subjected to colorimetry using a commercial measuring instrument from X-Rite at a viewing angle of 110° and an incident light angle of 45°. The measure of the opacity was the ratio of the lightness values at this measurement angle from the black side to the white side of the opacity chart. This parameter is plotted graphically in FIG. 1 for the various samples.

The closer the ratio of the measured lightnesses comes to a value of 1, the better the opacity.

Furthermore, laser granulometry was used for conventional determination of the size distribution of the aluminum effect pigments of the inventive and comparative examples. This was done using a Cilas 1064 instrument (Cilas, France). Tab. 1 shows the customary characteristic values of d₁₀ (fine fraction), d₅₀ (average), and d₉₀ (coarse fraction) of the corresponding cumulative undersize curve. These values represent volume-averaged values of equivalent spheres.

TABLE 1

| | | | | | | Gassing | |
|---|---|---|---|---|---|---|---|
| | | | | | | test 2 | |
| | | | | | | with | |
| | | | | | | (red) | |
| | | D₁₀/D₅₀/D₉₀ in | | Gassing | | iron | |
| | | [μm] | D₁₀/D₅₀/D₉₀ in | test 1 | | oxide | |
| | Aluminum | Cilas 1064 | [μm] | after | | after | |
| | pigment starting | starting | Cilas 1064 | 30 d in | | 56 d in | |
| Sample | material | pigment | coated pigment | [ml] | | [ml] | |
| Inventive | MEX 3580 | 7.5/12.1/18.7 | 8.1/13.0/19.3 | 0 | | 0 | |
| ex. 1 | (Eckart) | | | | | | |
| Comp. | MEX 3580 | 7.5/12.1/18.7 | 10.3/15.6/22.4 | <14 | d | 48 | d |
| ex. 1 | (Eckart) | | | | | | |
| Inventive | MEX 2156 | 9/17/28 | 9.6/17.5/27.9 | 0 | | 0 | |
| ex. 2 | (Eckart) | | | | | | |
| Comp. | MEX 2156 | 9/17/28 | 12.8/20.6/31.4 | 4 | ml | 4 | ml |
| ex. 2 | (Eckart) | | | | | | |
| Inventive | MEX 8154 | 9/20/32 | 9.6/20.1/33.1 | 0 | | 0 | |
| ex. 3 | (Eckart) | | | | | | |
| Comp. | MEX 8154 | 9/20/32 | 10.3/20.2/33.0 | 6.2 | | 2 | |
| Ex. 3 | (Eckart) | | | | | | |
| Inventive | Metalure | 4.3/11.4/19.2 | 4.5/11.7/19.6 | 0 | | 2.7 | ml |
| Ex. 4 | W-52012 | | | | | | |
| | IL | | | | | | |
| | (Avery | | | | | | |
| | Dennison) | | | | | | |
| Comp. | MEX 3580 | 7.5/12.1/18.7 | 13.5/31.0/61.8 | 1 | d | <1 | d |
| Ex. 4 | (Eckart) | | | | | | |
| Inventive | MEX 3580 | 7.5/12.1/18.7 | 8.5/14.1/19.6 | 27 | ml | 5 | ml |
| ex. 5 | (Eckart) | | | | | | |
| Inventive | MEX 3580 | 7.5/12.1/18.7 | 8.8/14.4/20.0 | 25 | ml | 4 | ml |
| Ex. 6 | (Eckart) | | | | | | |
| Comp. | Metalure | 4.3/11.4/19.2 | 4.5/11.6/19.3 | 4 | d | 26 | d |
| Ex. 5 | W-52012 | | | | | | |
| | IL | | | | | | |
| | (Avery | | | | | | |
| | Dennison) | | | | | | |

d: day(s). Number of days after which the permitted maximum amount of hydrogen evolved was exceeded.

From table 1 it is apparent that the difference in particle size distribution between each of the aluminum pigments used and the aluminum effect pigments then coated with the method of the invention (inventive examples 1 to 6) is only small. In the case of the comparative examples, the differences in size distribution between the aluminum effect pigments used and the aluminum effect pigments obtained after coating are substantially larger. This can be attributed to increasing agglomeration of the metallic effect pigment particles.

A small change in the particle size distribution means that the opacity of the aluminum effect pigments of the invention is not substantially adversely affected by coating, as compared with the conventionally coated aluminum effect pigments, and is therefore much better than for the coated pigments obtained in the comparative examples. The opacity of the aluminum effect pigments is illustrated in FIG. 1 for a number of inventive examples and comparative examples. Noteworthy therein in particular is the very poor opacity (in comparison to inventive example 1 and comparative example 1) for comparative example 4, which was carried out by a method based on WO 03/014228 A1.

Furthermore, as compared with aluminum effect pigments coated by a conventional sol-gel method, the aluminum effect pigments of the invention exhibit significantly improved gassing resistance, as evident from table 1. Here it is not the absolute values in the gassing tests that should be taken, but instead always the values of the inventive examples and comparative examples that correspond to one starting material should be compared. In the case of comparative examples 1, 4 and 5, the gassing test 1 had to be terminated after about 14 days and after about 1 day or 4 days, respectively, since the pigments had broken up completely, therefore failing to achieve a time of 30 days (30 d).

In gassing test 2 as well, the improved resistance of the pigment coatings of the invention was apparent, since as in gassing test 1 all of the inventive examples showed either no measurable evolution (0 ml in the case of examples 1 to 4) or acceptable evolution (examples 5-6) of hydrogen. In contrast, in the case of the comparative examples, the test had to be terminated after one day in one case. For the other comparative examples, hydrogen evolution of 2 to 7.3 ml was apparent after a test duration of 56 days. Even in those cases where the metallic effect pigments of the comparative examples are resistant to the gassing test, the pigments of the corresponding inventive examples are decidedly better.

The good balance between opacity (hiding power) and gassing stability of the aluminum effect pigments treated by the method of the invention with metal cations, presently Fe(III) ions and phosphorus-containing anions, preferably phosphate ions, and also of the aluminum effect pigments coated with the silicon oxide applied in two stages, can probably be attributed to the fact that, on the one hand, the corrosion stability—the stability with respect to corrosion—is increased in particular through the addition of Fe(III) ions and phosphate ions, for reasons which have so far not been understood, and, on the other hand, to the fact that, in spite of SiO₂ coating, a surprisingly large fine fraction is retained separately, and hence the opacity is significantly improved.

In a summarizing evaluation of the experimental results, it may be stated that the aluminum effect pigments coated by the method of the invention have significantly better performance properties in terms of opacity (hiding power), in conjunction with outstanding gassing stability, compared with aluminum effect pigments coated with SiO₂ by the conventional sol-gel method.

In terms of further optical properties such as luster and light/dark flop, the metallic effect pigments of the invention have not shown any detractions, in a variety of applications, when compared in each case with the pigments of the comparative examples.

The aluminum effect pigments produced in accordance with the invention can therefore be used with particular advantage not only in aqueous paint systems, aqueous inks, and aqueous printing-inks, but also in cosmetics, which are typically likewise water-containing.

1. A metallic effect pigment selected from the group consisting of platelet-shaped aluminum, platelet-shaped metallic pigments having a copper fraction of 60% to 100% by weight, and mixtures thereof, the metallic effect pigment having a coating of silicon oxide SiOₓ, where x is a number from 1 to 2,
wherein the coated metallic effect pigment comprises firstly metal cations and secondly at least one of phosphorus-containing anions and sulfur-containing anions, the metal cations and the at least one of the phosphorus-containing anions and the sulfur-containing anions being present in each case independently of one another on the metallic effect pigment surface, in the silicon oxide layer SiOₓ, or both on the metallic effect pigment surface and in the silicon oxide layer SiOₓ, and
the element ratio in atomic fractions of metal cation MC and phosphorus P to silicon Si, and metal cation MC and sulfur S to silicon Si, being defined in each case in accordance with the formulae (I) and (II)
100%×(MC+P)/Si (I)
100%×(MC+S)/Si
and being situated in total in a range from 0.5% to 35%.

2. The metallic effect pigment of claim 1,
wherein the metal cation MC is able to form a sparingly soluble salt in each case with the at least one of the phosphorus-containing anions and the sulfur-containing anions in aqueous solution within a pH from 5 to 8.

3. The metallic effect pigment of claim 1,
wherein the metal cations and the at least one of the phosphorus-containing anions and/or the sulfur-containing anions are present predominantly in the SiOx layer.

4. The metallic effect pigment of claim 1,
wherein the metal cations and the at least one of the phosphorus-containing anions and the sulfur-containing anions are present predominantly on the metallic effect pigment surface.

5. The metallic effect pigment of any of claim 1,
wherein the metal cations with the at least one of the phosphorus-containing anions and the sulfur-containing anions are present at least partly with one another in the form of a sparingly soluble salt.

6. The metallic effect pigment of claim 1,
wherein the at least one of the phosphorus-containing anions and the sulfur-containing anions are present predominantly on the metallic effect pigment surface and the metal cations are present predominantly in the SiOₓ layer.

7. The metallic effect pigment of claim 1,
wherein the metal cations are present predominantly on the metallic effect pigment surface and the at least one of the phosphorus-containing anions and the sulfur-containing anions are present predominantly in the SiOₓ layer.

8. The metallic effect pigment of claim 1,
wherein the metal cations for the phosphorus-containing anions are selected from the group consisting of Ag(I), Cu(II), Cd(II), Cr(III), Co (II), Pb (II), Hg (I), Hg (II), Mg (II), Al (III), Zn (II), Sn (II), Ca (II), Sr (II), Ba (II), Mn (II), Bi(III), Zr(IV), Ni(II), Fe(II), Fe(III), and mixtures thereof, and also mixtures thereof with ammonium ions.

9. The metallic effect pigment of claim 1,
wherein the metal cations for sulfate ions as sulfur-containing anions are selected from the group consisting of Ag(I), Sb(III), Ca (II), Ba (II), Sr (II), Pb (II), Fe (III), and mixtures thereof.

10. The metallic effect pigment of claim 1,
wherein the metal cations for sulfide anions as sulfur-containing anions are selected from the group consisting of Ag(I), Sb(III), Bi (III), Cd (II), Co (II), Cu (II), Ca (II), Ba: (II), Pb (II), Mn(II), Ni(II), Sn(II), Sn(IV), Zn(II), Fe (II), and mixtures thereof.

11. The metallic effect pigment of claim 1,
wherein the SiOₓ layer is present in amounts of 2% by weight to 25% by weight, calculated as SiO₂ and based on the weight of the metallic effect pigment.

12. The metallic effect pigment of claim 1,
wherein the metallic effect pigments have a noncalcined silicon oxide layer.

13. The metallic effect pigment of claim 1,
wherein the metallic effect pigments are present in the form of powder, dry product or paste, and at least one of (1) in the silicon oxide layer; (2) on the silicon oxide layer; and (3) in the solvent of the paste there is
0.01%-1% by weight of at least one of organic acid and inorganic acid and
0.01%-1% by weight of at least one of organic base and inorganic base,
the percent by weight figures being based on the total weight of the pigment.

14. The metallic effect pigment of claim 13,
wherein the organic acid contains 1 to 8 C atoms.

15. The metallic effect pigment of claim 13,
wherein the base is an amine.

16. The metallic effect pigments of claim 1,
wherein the metallic effect pigments are present in an entirety, the entirety comprising at least three silicon oxide-coated metallic effect pigments whose d₅₀ diameters each differ by 2 to 6 μm, the smallest d₅₀ of a metallic effect pigment from the entirety being not more than 5 μm.

17. The metallic effect pigments of claim 16,
wherein the entirety comprises at least four silicon oxide-coated metallic effect pigments whose d₅₀ diameters each differ by 3 to 5 μm.

18. A method for producing silicon oxide-coated metallic effect pigments of claim 1,
wherein the method comprises the following steps:
(a) applying silicon oxide to the metallic effect pigments, with reaction of at least one of alkoxysilane(s) and silicon halide(s) in organic solvent with water in the presence of at least one of acid and base as catalyst,
(b) adding metal cations and at least one of phosphorus-containing anions and/or sulfur-containing anions to the metallic effect pigments before or during step (a), with the metal cations and the at least one of the phosphorus-containing anions and the sulfur-containing anions being taken up (1) onto the metallic effect pigment surface, (2) into the silicon oxide layer; or (3) both onto the metallic effect pigment surface and into the silicon oxide layer,
(c) optionally applying a surface modification to the silicon oxide surface.

19. The method for producing silicon oxide-coated metallic effect pigments of claim 18,
wherein the method comprises the following steps:
(a) applying silicon oxide to the metallic effect pigments, with reaction of at least one of alkoxysilane(s) and silicon halide (s) in organic solvent with water in the presence of at least one of acid and base as catalyst,
(b) adding metal cations and at least one of phosphorus-containing anions and sulfur-containing anions to the metallic effect pigments before or during step (a), with the metal cations and at least one of the phosphorus-containing anions and the sulfur-containing anions being taken up predominantly into the silicon oxide layer,
(c) optionally applying a surface modification to the silicon oxide surface.

20. The method for producing silicon oxide-coated metallic effect pigments of claim 18,
wherein the method comprises the following steps:
(a) adding metal cations and at least one of phosphorus-containing anions and sulfur-containing anions to the metallic effect pigments, with the metal cations and the at least one of the phosphorus-containing anions and the sulfur-containing anions being taken up predominantly onto the metallic effect pigment surface,
(b) applying silicon oxide to the metallic effect pigments treated according to step (a), with reaction of at least one of alkoxylsilane(s) and silicon halide(s) in organic solvent with water in the presence of at least one of acid and base as catalyst,
(c) optionally applying a surface modification to the silicon oxide surface.

21. The method for producing silicon oxide-coated metallic effect pigments of claim 18,
wherein the method comprises the following steps:
(a) applying silicon oxide to the metallic effect pigments, with reaction of at least one of alkoxysilane(s) and silicon halide(s) in organic solvent with water in the presence of at least one of acid and base as catalyst,
(b) adding at least one of phosphorus-containing anions, sulfur-containing anions, and metal cations to the metallic effect pigments before or during step (a), with the at least one of the phosphorus-containing anions and the sulfur-containing anions being taken up predominantly onto the metal surface, and with metal cations being taken up predominantly into the silicon oxide layer,
(c) optionally applying a surface modification to the silicon oxide surface.

22. The method for producing silicon oxide-coated metallic effect pigments of claim 18,
wherein the method comprises the following steps:
(a) applying silicon oxide to the metallic effect pigments, with reaction of at least one of alkoxysilane(s) and silicon halide(s) in organic solvent with water in the presence of at least one of acid and base as catalyst,
(b) adding at least one of phosphorus-containing anions, sulfur-containing anions, and metal cations to the metallic effect pigments before or during step (a), with metal cations being taken up predominantly onto the metal surface, and with the at least one of the phosphorus-containing anions and the sulfur-containing anions being taken up predominantly into the silicon oxide layer,
(c) optionally applying a surface modification to the silicon oxide surface.

23. The method of claim 18,
wherein the adding of the at least one of the phosphorus-containing and the sulfur-containing anions on the one hand and of the metal cations on the other hand takes place in separate steps, simultaneously or in succession.

24. The method of claim 18,
wherein the applying of silicon oxide, with reaction of alkoxysilane(s) in organic solvent with water, is catalyzed by acids and bases,
(i) the reaction being carried out in a first step with addition of acid and in a second step with addition of base, or
(ii) the reaction being carried out in a first step with addition of base and in a second step with addition of acid.

25. The method of claim 18,
wherein the acid is an organic acid and contains 1 to 8 C atoms.

26. The method of claim 18,
wherein the method is conducted as a one-pot reaction.

27. A process for producing a pigmented cosmetic, plastic or coating composition, comprising introducing the metallic effect pigment of claim 1 into a cosmetic, plastic or coating composition to form a pigmented cosmetic, plastic or coating composition.

28. An article
comprising the metallic effect pigment of claim 1.

29. The process according to claim 27, wherein the coating composition is selected from the group consisting of ink, printing-ink, paint, and powder coating material.
